(19) United States
(12) Patent Application Publication
(10) Pub. No.: US 2026/0065710 A1
(43) Pub. Date: Mar. 5, 2026

(54) Sensors and Methods

(71) Applicant: Touch Biometrix Limited, St Asaph (GB)

(72) Inventors: Toru SAKAI, St Asaph (GB); Wilhelmus VAN LIER, St Asaph (GB); Henricus DERCKX, St Asaph (GB)

(21) Appl. No.: 19/103,189

(22) PCT Filed: Aug. 14, 2023
(86) PCT No.: PCT/GB2023/052135

§ 371 Date: Feb. 11, 2025

(30) Foreign Application Priority Data
Aug. 12, 2022 (GB) 2211841.8
Aug. 12, 2022 (GB) 2211848.3
Aug. 12, 2022 (GB) 2211850.9
Apr. 18, 2023 (GB) 2305664.1
Apr. 18, 2023 (GB) 2305678.1

(51) Int. Cl. G06V 40/13; A61B 5/1172

(52) U.S. Cl. CPC G06V 40/13; A61B 5/1172; A61B 2562/125

(57) ABSTRACT

Disclosed herein is a skin contact sensor comprising a sensor array of sensor pixels, wherein: each sensor pixel comprises at least one thin film transistor, TFT, and a sensing electrode; a top surface of the sensor array provides a contact surface for contacting by an object to be sensed; and each sensor pixel is formed of a plurality of layers including an optical colour filter layer arranged to filter one or more colours of light.

### TECHNICAL FIELD

The present disclosure relates to the field of capacitive biometric skin contact sensors, as well as methods of designing capacitive biometric skin contact sensors, and methods of designing capacitive biometric skin contact sensors.

### BACKGROUND

Biometric skin sensors typically utilise optical sensors. Such optical sensors rely on being able to image a user's skin. The optical sensor will obtain an image of skin interacting with a sensing surface of the sensor. Typically this will involve the person placing their fingertip on the sensing surface so that image data of their fingertip is obtained. That image data is compared against reference data (i.e. known fingertip data) to try to identify that person. If the image data of the person's fingertip matches reference data of a known authorised user, then person with their fingertip on the sensing surface is an authorised user. In particular, the optical sensor will identify if the contours of the person's skin (ridges and valleys in their skin) are the same as those of the authorised user. Such optical biometric sensors may be included in electronic devices or other suitable equipment to provide biometric authentication. However, for such optical biometric sensors to work, it is essential that they can obtain images of a person's skin on the sensing surface. As a result, for such an optical sensor to be used in any given device, that device will need to include a transparent region that is aligned with the contact surface. That way, a user interacting with the device can place their finger on the transparent portion of the device, and the optical sensor may image their finger for biometric authentication. The inclusion of such optical biometric sensors in these devices therefore places constraints as to the design and appearance of those electronic devices.

### SUMMARY

Aspects of the disclosure are set out in the independent claims and optional features are set out in the dependent claims. Aspects of the disclosure may be provided in conjunction with each other, and features of one aspect may be applied to other aspects.

In an aspect, there is provided a skin contact sensor comprising a sensor array of sensor pixels, wherein: each sensor pixel comprises at least one thin film transistor, TFT, and a sensing electrode; a top surface of the sensor array provides a contact surface for contacting by an object to be sensed; and each sensor pixel is formed of a plurality of layers including an optical colour filter layer arranged to filter one or more colours of light.

The sensor may be a capacitive sensor. The sensing electrode may be a capacitive sensing electrode. The sensor may comprise a biometric sensor. For example, the sensor may comprise a capacitive biometric skin contact sensor. Embodiments may enable skin contact sensing (e.g. capacitive biometric skin contact sensing) to be provided by a non-transparent sensor. The sensor (e.g. the capacitive sensor) may obtain biometric skin contact data for the object to be sensed, and the sensor will have a colour filter layer deposited thereon. Due to colour filtering by the colour filter layer, the sensor will appear coloured to a user of the sensor, where that colour is based on properties of the colour filter chosen for each sensor pixel in the sensor array. The provision of a colour filter layer may enable greater design freedom for the appearance of the sensor (e.g. the biometric sensor), while still enabling the sensor to perform skin contact sensing for a user interacting with that sensor (e.g. capacitive biometric skin contact sensing).

Sensors of the present disclosure may be configured so that light travelling from the sensor towards a user of the sensor will travel through the optical colour filter layer of the sensor. The optical colour filtering layer may be configured to provide optical colour filtering of that light so that the sensor appears coloured to the user interacting with the sensor (e.g. where the particular appearance is governed by one or more properties of the chosen colour filter). The sensor may be a reflective light colour filtering sensor, a transmissive light colour filtering sensor or a combination thereof (e.g. in which both reflective and transmissive colour filtering is provided). For a reflective light colour filtering sensor, the sensor may comprise at least one optically reflective component configured to reflect light back towards the user (and through the colour filter). For the transmissive light colour filtering sensor, the sensor may be provided with a backlight which is arranged to direct light through the colour filter of the sensor pixel and to the user. In either case, light will be colour filtered as it passes through the optical colour filter layer, thereby causing the sensor to appear coloured to the user. A combined transmissive and reflective light colour filtering sensor may be provided in which at least some light is reflected (e.g. from an optically reflective portion of the sensor) which will travel through the colour filter, and at least some light will be transmitted (e.g. from the backlight) which will travel through the colour filter.

The plurality of layers may include an optically reflective layer located below the optical colour filter layer and comprising an optically reflective element. For example, there may be one or more different layers separating the optically reflective layer from the colour filter layer. The optically reflective element may comprise an electrical conductor. For example, electrically conductive and optically reflective material may be used for the electrical conductor. The optically reflective layer may comprise a metallization layer in which the metal is optically reflective. It may comprise multiple such layers.

The sensing electrode (e.g. the capacitive sensing electrode) may provide the optically reflective element for each sensor pixel. For each sensor pixel, the optically reflective layer may be a first optically reflective layer, and the sensor pixel may also include a second optically reflective layer comprising an optically reflective element. The optical colour filter layer may be located above the reflective elements of both reflective layers. Each sensor pixel may comprise an electrical shield layer comprising an electrical shield. The electrical shield layer may provide a first plate of a reference capacitor for the sensor pixel and the capacitive sensing electrode may provide a second plate of the reference capacitor. As another example, a reference capacitor may be provided by a plate in a first layer (e.g. a gate or source/drain layer) and a plate in a second layer (e.g. the other of the gate or source/drain layer). Other layers could be used to provide reference capacitor plates. The sensing electrode (e.g. capacitive sensing electrode) may be located above the electric shield. Both the (capacitive) sensing electrode and the electric shield may provide optically reflective elements for each sensor pixel. The colour filter layer may be located above the (capacitive) sensing electrode and the electric shield.

Some or all electrically conductive components of the sensor pixel may provide optically reflective elements. Each electrically conductive component of the sensor pixel may be made of an optically reflective material, such as an aluminium alloy (other example materials also include molybdenum or titanium). For each sensor pixel, the optical colour filter layer may be provided on a top surface of the sensing electrode (e.g. the capacitive sensing electrode). For each sensor pixel, a passivation layer may be provided on a top surface of the (capacitive) sensing electrode. The optical colour filter layer may be provided on a top surface of the passivation layer. For each sensor pixel, a hard coat may be provided on top of the sensor pixel. The optical colour filter layer may be provided on a top surface of the hard coat or it may be provided on a lower surface (e.g. with the hard coat provided on top of the colour filter layer). For example, the passivation layer may be included (above the sensing electrode), an the optical colour filter layer may be provided on a top surface of the passivation layer, with a hard coat then provided on top of that colour filter layer. A hydrophobic coating may be provided on a top surface of the sensor pixel. For example, the hydrophobic coating may be provided above the hard coat.

For at least some of the sensor pixels, an aperture ratio for the colour filter may be varied to provide a selected property for the colour filtering provided by said pixels. For at least some of the sensor pixels, the colour filter layer for the sensor pixel may include a first colour filter and a second colour filter. The first colour filter may be for a different colour to the second colour filter. The first colour filter may be transparent (e.g. a ‘white’ filter), or opaque (e.g. a ‘black’ filter). A ‘white’ filter may comprise a filter with no pigments, such as an organic resin without any pigments. Such a ‘white’ filter may therefore be (e.g. substantially) transparent (e.g. rather than being coloured white per se.). In other words, use of a ‘white’ filter may provide maximum transmission of light therethrough (e.g. maximum transparency). For example, the first colour filter may be white or black. The second colour filter may another colour, such as red, green or blue. A ratio of the first colour filter to the second colour filter on the sensor pixel may be selected to a provide a selected colour property to the sensor pixel. For example, the area of the sensor pixel covered by the first colour filter (as compared to the second colour filter) may be selected to provide a selected property to the colour of the sensor pixel. The selected colour property may comprise a brightness and/or a darkness. In other words, each sensor pixel may be designed to provide a certain colour for the colour filtering, e.g. where that colour contains a desired grayscale effect for that particular colour.

For a reflective colour filtering sensor (and/or the combination of transmissive and reflective), the sensor may include an optically reflective layer located below the optical colour filter layer. The optically reflective layer may comprise an optically reflective element. Any non-transparent components of the sensor pixel in layers between the optically reflective layer and the colour filter layer may be spatially arranged to inhibit blocking (e.g. attenuation) of light travelling between the optical reflective layer and the optical colour filter layer. For example, said non-transparent components of the sensor pixel may be arranged to be laterally offset from an area of the sensor underneath the colour filter. For each sensor pixel, non-transparent components of the sensor pixel located above the optically reflective layer may be spatially arranged to inhibit blocking (e.g. attenuation) of light travelling between the optical reflective layer and the optical colour filter layer. For example, said non-transparent components of the sensor pixel may be laterally offset from a region of the sensor pixel covered by the optical colour filter. Said non-transparent components may be aligned with regions of the sensor array in between adjacent (capacitive) sensing electrodes. For example, said non-transparent components may be located underneath regions of the sensor pixel which are not covered by a colour filter.

For a transmissive colour filtering sensor (and/or the combination of transmissive and reflective), the sensor may be arranged to be backlit by a transmitting element located below the optical colour filter layer, and wherein any non-transparent components of the sensor pixel in layers between the transmitting element and the colour filter layer are spatially arranged to inhibit blocking (e.g. attenuation) of light travelling between the transmitting element and the optical colour filter layer. For example, said non-transparent components of the sensor pixel may be arranged to be laterally offset from an area of the sensor underneath the colour filter. For each sensor pixel, non-transparent components of the sensor pixel located above the transmitting elements may be spatially arranged to inhibit blocking (e.g. attenuation) of light travelling between the transmitting element and the optical colour filter layer. For example, said non-transparent components of the sensor pixel may be laterally offset from a region of the sensor pixel covered by the optical colour filter. Said non-transparent components may be aligned with regions of the sensor array in between adjacent (capacitive) sensing electrodes. For example, said non-transparent components may be located underneath regions of the sensor pixel which are not covered by a colour filter.

For each sensor pixel, the (capacitive) sensing electrode may be at least partially optically transparent. For example, a reflective element (and/or a backlight) may be provided beneath the (capacitive) sensing electrode (and light may pass therefrom and through the partially transparent capacitive sensing electrode). For example, a colour filter may be located beneath the (capacitive) sensing electrode (or this could be located above the capacitive sensing electrode). Each sensor pixel may comprise at least one of: (i) an optically reflective electric shield; (ii) optically reflective source and/or drain conductive elements; (iii) an optically reflective gate conductive element; (iv) a substrate onto which the sensor pixel is built, wherein a surface of the substrate is optically reflective, optionally wherein the surface is a bottom surface of the substrate; thereby to provide the optically reflective element of the sensor pixel.

In an aspect, there is provided an apparatus comprising any sensor of the present disclosure. The apparatus includes a light transmitting element, wherein the light transmitting element is arranged beneath the colour filter layer of the sensor pixels of the sensor. The light transmitting element may be part of the sensor or may be provided by a separate component to the sensor. The apparatus may include a controller configured to control operation of the light emitting element. For example, the controller may be configured to selectively activate the transmitting element to display information to a user of the sensor, where the information is displayed using the one or more colour filters of the sensor array.

In an aspect, there is provided a method of manufacturing a sensor, the sensor comprising a sensor array of sensor pixels, wherein a top surface of the sensor array provides a contact surface for contacting by an object to be sensed, and wherein each sensor pixel comprises at least one thin film transistor, TFT, and a sensing electrode. The method comprises: for each sensor pixel, providing a plurality of layers for that sensor pixel including an optical colour filter layer. The method may be a method of manufacturing a capacitive biometric sensor. The sensing electrode may be a capacitive sensing electrode. The sensor may be a capacitive biometric skin contact sensor. Providing the optical colour filter layer for the sensor pixels of the array may comprise use of a photolithography method. Providing the plurality of layers may comprise providing the (capacitive) sensing electrode for the sensor pixel. The (capacitive) sensing may be optically reflective and provided in a layer beneath the optical colour filter layer, thereby to provide an optically reflective layer beneath the colour filter layer. Providing the optical colour filter layer for each sensor pixel may comprise depositing an optical colour filter above the (capacitive) sensing electrode.

In an aspect, there is provided a method of designing a sensor, the sensor comprising a sensor array of sensor pixels, wherein a top surface of the sensor array provides a contact surface for contacting by an object to be sensed, wherein each sensor pixel comprises at least one thin film transistor, TFT, and a sensing electrode, and wherein each sensor pixel is formed of a plurality of layers including an optical colour filter layer arranged to filter one or more colours of light. The method comprises: obtaining an indication of a selected appearance for the top surface of the sensor; and selecting at least one optical property for the optical filter of each sensor pixel of the array based on the selected appearance for the top surface of the sensor. The selected appearance for the top surface of the sensor may comprise a spatial distribution of one or more colours across the sensor array. The method may comprise a method of designing a capacitive sensor, e.g. a capacitive biometric skin contact sensor. The sensing electrode may comprise a biometric sensing electrode. Selecting the at least one optical property for the optical filter of each sensor pixel of the array may comprise selecting a colour for said optical filter, wherein said colour may be selected according to the spatial distribution of colours for the sensor array. For example, one or more pixel dithering techniques could be applied to vary particular colours for each optical filter.

In aspects of the present disclosure, sensing functionality (e.g. capacitive biometric skin contact sensing functionality) is provided by a sensor which also includes an optically reflective layer and a colour filter (for filtering reflected light from a reflective element in the optically reflective layer). The optically reflective layer may be arranged to provide diffuse reflection of light incident on the sensor from above. For example, the sensor may be configured so that such diffuse reflected light may pass through the colour filter of the sensor pixel (thereby to provide colour filtered diffuse reflected light). The (capacitive) sensing electrode may provide the (diffuse) optically reflective layer. A top surface of the (diffuse) optically reflective layer may be uneven. The top surface of the optically reflective layer may include at least one peak and at least one trough. The top surface of the optically reflective layer may define a series of islands of material (e.g. where the material of the top surface is elevated relative to other portions of the optically reflective layer). There may be a plurality of peaks and troughs for each sensor pixel. The peaks and troughs may be arranged in a selected pattern across the sensor pixel. The selected pattern may be a random pattern and/or a non-regular pattern. The peaks may be provided by islands of material, and wherein at least one of: (i) a shape of the islands, (ii) a cross-sectional profile of the islands, (iii) a spatial arrangement and/or distribution of the islands is chosen to provide a selected appearance for the sensor. The peaks and troughs may be arranged in a regular repeating pattern across the sensor pixel.

Adjacent peaks may be separated by at least 2, e.g. at least 5 (e.g. at least 10) microns. A difference in height between peak and trough may be at least 1 micron. The optically reflective layer may be provided by an optically reflective and electrically conductive layer in the pixel. The electrically conductive layer may be deposited onto a textured layer, wherein the textured layer may have an uneven surface thereby to impart a corresponding uneven surface to the optically reflective layer disposed thereon. The textured layer may be an insulator layer. The optically reflective layer may be deposited onto a user facing surface of the textured layer. The optically reflective layer may have a substantially uniform thickness across the sensor pixel. Any electrically conductive components above the optically reflective layer may be provided by transparent electrical conductors. Each sensor pixel may comprise an electrical shield layer comprising an electrical shield, and wherein the (e.g. capacitive) sensing electrode may be located above the electric shield. At least one of the electrical shield layer and the (e.g. capacitive) sensing electrode may provide a said optically reflective layer for the sensor. Aspects may also provide methods of manufacturing such a sensor, as well as methods of calibrating such a sensor.

Aspects of the present disclosure may include an optically reflective layer arranged to provide diffuse reflection of light without a colour filter being included. For example, in an aspect, there is provided a skin contact sensor (e.g. a capacitive biometric skin contact sensor) comprising a sensor array of sensor pixels, wherein: each sensor pixel comprises at least one thin film transistor, TFT, and a sensing electrode; a top surface of the sensor array provides a contact surface for contacting by an object to be sensed; and each sensor pixel is formed of a plurality of layers including an optically reflective layer arranged to provide diffuse reflection of light incident on the sensor from above. The sensor may comprise a capacitive biometric skin contact sensor. The sensing electrode may comprise a capacitive sensing electrode.

The (e.g. capacitive) sensing electrode may provide the optically reflective layer. A top surface of the optically reflective layer may be uneven. The top surface of the optically reflective layer may include at least one peak and at least one trough. There may be a plurality of peaks and troughs for each sensor pixel. The top surface of the optically reflective layer may define a series of islands of material (e.g. where the material of the top surface is elevated relative to other portions of the optically reflective layer). There may be a plurality of peaks and troughs for each sensor pixel. The peaks and troughs may be arranged in a selected pattern across the sensor pixel. The selected pattern may be a random pattern and/or a non-regular pattern. The peaks may be provided by islands of material, and wherein at least one of: (i) a shape of the islands, (ii) a cross-sectional profile of the islands, (iii) a spatial arrangement and/or distribution of the islands is chosen to provide a selected appearance for the sensor. The peaks and troughs may be arranged in a regular repeating pattern across the sensor pixel.

Adjacent peaks may be separated by at least 2, e.g. at least 5 (e.g. at least 10) microns. A difference in height between peak and trough may be at least 1 micron. The optically reflective layer may be provided by an optically reflective and electrically conductive layer in the pixel. The electrically conductive layer may be deposited onto a textured layer, wherein the textured layer may have an uneven surface thereby to impart a corresponding uneven surface to the optically reflective layer disposed thereon. The textured layer may be an insulator layer. The optically reflective layer may be deposited onto a user facing surface of the textured layer. The optically reflective layer may have a substantially uniform thickness across the sensor pixel. Any electrically conductive components above the optically reflective layer may be provided by transparent electrical conductors. Each sensor pixel may comprise an electrical shield layer comprising an electrical shield, and wherein the (e.g. capacitive) sensing electrode may be located above the electric shield. At least one of the electrical shield layer and the (e.g. capacitive) sensing electrode may provide a said optically reflective layer for the sensor.

In an aspect, there is provided a method of manufacturing a skin contact sensor, the sensor comprising a sensor array of sensor pixels, wherein a top surface of the sensor array provides a contact surface for contacting by an object to be sensed, and wherein each sensor pixel comprises at least one thin film transistor, TFT, and a sensing electrode, the method comprising: for each sensor pixel, providing a plurality of layers for that sensor pixel including an optically reflective layer arranged to provide diffuse reflection of light incident on the sensor from above. The method may comprise a method of manufacturing a capacitive sensor, e.g. a capacitive biometric skin contact sensor. The sensing electrode may comprise a capacitive sensing electrode. Providing the optically reflective layer may comprise: texturing a textured layer of the sensor pixel thereby to provide an uneven surface to the textured layer of the sensor pixel; and depositing an optically reflective material onto the uneven surface of the textured layer, thereby to provide an uneven top surface of the optically reflective material. The textured layer may be an electrical insulator layer. The optically reflective layer may be an electrically conductive and optically reflective layer. The optically reflective layer may provide the sensing electrode, e.g. the capacitive sensing electrode, for the sensor pixel.

In an aspect, there is provided a method of calibrating a skin contact sensor (e.g. a capacitive biometric skin contact sensor), the sensor comprising a sensor array of sensor pixels, wherein a top surface of the sensor array provides a contact surface for contacting by an object to be sensed, wherein each sensor pixel comprises at least one thin film transistor, TFT, and a sensing electrode, and wherein each sensor pixel is formed of a plurality of layers including an optically reflective layer arranged to provide diffuse reflection of light incident on the sensor from above, the method comprising: operating a sensor pixel to obtain data (e.g. capacitance data) for a reference parameter (e.g. a reference capacitance); and storing calibration data for that sensor pixel, wherein the stored calibration data is based on the obtained data (e.g. the obtained capacitance data) for the reference parameter (e.g. the reference capacitance). The method may comprise a method of calibrating a capacitive biometric skin contact sensor. The sensing electrode may comprise a capacitive sensing electrode. The reference parameter (e.g. the reference capacitance) may comprise a black noise image. The calibration data may comprise an indication of a difference between the obtained parameter (e.g. the obtained capacitance) data and a parameter (e.g. a capacitance) associated with the black noise image.

Aspects of the present disclosure may utilise two or more electrically conductive materials to provide an electrically conductive and optically reflective element of the sensor. The combination of materials used to provide that optically reflective element may be chosen to provide a selected overall reflectance for the optically reflective element (e.g. a ratio of first to second materials used may be controlled to provide a selected reflectance for the element).

The optically reflective element may comprise a first material and a second material. The first material may have a different optical property to the second material. The first material may be electrically conductive and optically reflective. The second material may be electrically conductive and either: (i) at least partially optically transparent, or (ii) optically reflective with a different reflectance to the first material. The combination of the first material and the second material may be selected to provide a selected appearance property for optically reflective element. A first electrically conductive element in a first subregion of the sensor array may be provided by a combination of both a first material and a second material. The combination may be selected to provide a selected appearance property for the first subregion of the sensor array. The selected appearance property for the first subregion may comprise an overall reflectance value for the first subregion. The first material may be electrically conductive and optically reflective. The second material may be electrically conductive and either: (i) at least partially optically transparent, or (ii) optically reflective with a different reflectance to the first material.

In an aspect, there is provided a touch sensor (e.g. a capacitive touch sensor, e.g. a capacitive biometric skin contact sensor) comprising a sensor array of sensor pixels, wherein a first electrically conductive element in a first subregion of the sensor array is provided by a combination of both a first material and a second material. The first electrically conductive element may comprise a sensing electrode, such as a capacitive sensing electrode. Each pixel of the sensor array may comprise a sensing electrode (e.g. a capacitive sensing electrode), and wherein at least some of the (e.g. capacitive) sensing electrodes are formed of two electrically conductive materials.

In an aspect, there is provided a sensor (e.g. a capacitive sensor) comprising an array of sensor pixels, each sensor pixel comprising a sensing electrode (e.g. a capacitive sensing electrode) and at least one thin film transistor, TFT, wherein: a top surface of the sensor is arranged for contacting by an object to be sensed; each sensor pixel is provided by a multi-layered pixel stack comprising a plurality of electrically conductive layers; and a first electrically conductive layer of the pixel stack of a first sensor pixel is provided by a first area of a first material and a second area of a second material. The first material may be electrically conductive and optically reflective. The second material may be electrically conductive and either: (i) at least partially optically transparent, or (ii) optically reflective with a different reflectance to the first material. The second area of material may be provided on a top surface of the first area of material. The second area of material may not cover the entirety of the top surface of the first area of material. The second area of material may be thinner than the first area of material. The portion of the first area of material not covered by the second area of material may differ between different sensor pixels in the array. A size of the portion of the first area of material not covered by the second area of material may be selected to provide a selected reflectance property. For each said sensor pixel, a ratio of: (i) the size of the portion of the first area of material not covered by the second area of material, to: (ii) the size of the portion of the second area of material, may be selected to provide a selected reflectance for that sensor pixel. The first electrically conductive layer of the first sensor pixel may provide the sensing electrode (the capacitive sensing electrode) for said first sensor pixel.

In an aspect, there is provided a method of manufacturing a touch sensor (e.g. a capacitive touch sensor) comprising a sensor array of sensor pixels, wherein the method comprises: choosing a combination of both a first material and a second material to be used to provide a first electrically conductive element in a first subregion of the sensor array so that an overall reflectance for the first subregion will be at a selected reflectance value; and providing the first electrically conductive element according to the chosen combination of the first and second materials.

Embodiments may enable the provision of a touch sensor which is configured to provide selected optical reflectance properties while still being able to function as intended. That is, the first sub-region of the sensor array may be configured to provide both touch sensing (e.g. capacitive touch sensing) and also a selected reflectance property for light incident on that sub-region. For example, each sub-region of the sensor array may also provide its own selected reference value, and the reflectance values for all of the different sub-regions may be selected to give rise to a chosen overall appearance for the sensor array. As such, embodiments may enable the provision of a sensor which will provide touch sensing (e.g. capacitive touch sensing) whilst also being customisable in appearance (e.g. to provide a desired reflectance for the sub-region(s) of the sensor array).

The first material may be electrically conductive and optically reflective. The second material may be electrically conductive and either: (i) at least partially optically transparent (referred to as a “third material” in the description), or (ii) optically reflective with a different reflectance to the first material (referred to as a “second material” in the description). For example, the second material may be more optically reflective than the first material. The second material may provide a bright reflector and the first material may provide a dark reflector. Methods may comprise modulating an area of the first electrically conductive element provided by the second material to provide the selected reference value for the first subregion.

A user may interact with a contact surface of the sensor. For example, the contact surface may be the surface for which the sensor is configured to provide contact sensing. The contacting sensing may comprise obtaining an indication of a portion of the contact surface which has been contacted. Contact sensing may comprise performing biometric skin contact sensing for sensing properties of skin contacting the contact surface (e.g. for biometric analysis thereof). The method may comprise depositing the second material onto a user-facing surface of the first material. The contact surface may be provided on a user-side of the sensor. The user-side of the sensor may also be referred to as a ‘top’ or ‘upper’ side of the sensor. The method may comprise modulating an area of the user-facing (e.g. top) surface of the first material covered by the second material to provide the selected reflectance value for the first subregion. In other words, the method may comprise modulating how much of the first material (e.g. the top surface thereof) is covered by the second material, e.g. to control how much light incident on the conductive element will be incident on the first material or the second material. By providing a greater coverage of second material (i.e. by having a larger area of second material overlying the first material), more light incident on the conductive element will be incident on the second material. Where the second material is reflective (“second material”), that means more light will be reflected by the second material. Where the second material is transparent (“third material”), that means more light which is ultimately reflected by the first material will have passed through the second material. Where a third material is used, the first material may be provided by a relatively high reflectance material, such as an aluminium alloy or a silver-based material.

The method may comprise removing some of the second material deposited onto the user-facing surface of the first material so as to provide a selected area of the user-facing surface of the first material which is covered by the second material. For example, the method may comprise initially having two (full) layers of conductive material (first and second material) and removing some of the second material to control how much second material will be included for each conductive element. The method may further comprise removing some of the first material so that the remaining islands of first material provide separate conductive elements for the array (e.g. capacitive sensing electrodes).

The second material may be more optically reflective than the first material. Choosing the combination of the first and second materials may comprise selecting an amount of second material needed to increase the overall reflectance for the first subregion from: (i) a reflectance corresponding to the first electrically conductive element being at the first reflectance, to (ii) the selected reflectance value. For example, choosing the combination may comprise choosing a proportion of a reflective area of the conductive element which is to be provided by second material (e.g. and increasing that proportion to increase the reflectance for the element). The reflective area of each conductive element may comprise a visible upper surface of the electrically conductive element. That is, the reflective area may comprise a top (i.e. user-facing) surface of the second material and a top (i.e. user-facing) surface of any first material which is not covered by second material. In other words, the reflective area of each conductive element may comprise the area from which light incident on that conductive element will reflect. That light may be incident from a user-facing side of the sensor (e.g. the light may be incident on the element from above). The available surface of the conductive element from which that incident light may reflect (e.g. when viewed from the user-side, e.g. above) may provide the reflective area of the element. A portion of that reflective area may be provided by second material (e.g. which may be on top of the first material) and a portion of that reflective area may be provided by first material (e.g. which does not have any second material on top of it). Providing a greater value for the overall reflectance for the first subregion may comprise providing an increased amount of second material.

The first electrically conductive element may be one of: (i) a sensing electrode (e.g. a capacitive sensing electrode) of one sensor pixel in the sensor array, and (ii) an electrical shield layer for one or more sensor pixels in the sensor array. For example, each sensing electrode (e.g. capacitive sensing electrode) and/or each shield may be used to provide an optically reflective element of the sensor array. For each subregion of the sensor array, the method may comprise: choosing a combination of first and second materials to be used to provide an electrically conductive element in said subregion of the sensor array so that an overall reflectance for said subregion will be at a selected reflectance value; and providing the electrically conductive element in said subregion according to the chosen combination of the first and second materials for said subregion. In at least one subregion, the electrically conductive element may either be entirely provided (e.g. covered) by the first material or entirely provided (e.g. covered) by the second material. For example, in said at least one subregion, the reflectance may be that associated with only one of the first or second material being present (e.g. it may either represent maximum reflection brightness or minimum reflection brightness).

The method may further comprise providing a colour filter layer above the first electrically conductive element. The method may comprise providing a colour filter layer above some or all of the electrically conductive elements of the entire sensor array. For example, each electrically conductive and optically reflective element may have a colour filter layer above it. The colour filter layer may be provided above the optically reflective element(s) of the sensor array. For example, the colour filter layer may be provided in one of the higher layers of the pixel stack, e.g. so that reflected light from the sensor array will pass through the colour filter layer. The colour filter layer may be arranged to provide optical colour filtering of light reflected from the optically reflective element (e.g. as well as filtering of light passing through the colour filter on the way to being incident on the optically reflective element). One or more colour(s) may be chosen for each colour filter, and a selected reflectance value may be chosen for each optically reflective element to give rise to a desired colour appearance for that sub-region of the sensor array. For example, the brightness (e.g. shade) of the resulting colour may be controlled by modulating an amount of second material present for providing the optically reflective element.

In an aspect, there is provided a touch sensor (e.g. a capacitive touch sensor) comprising a sensor array of sensor pixels, wherein: a first electrically conductive element in a first subregion of the sensor array is provided by a combination of both a first material and a second material; and the combination is selected to provide a selected appearance property for the first subregion of the sensor array.

The first material may be electrically conductive and optically reflective. The second material may be electrically conductive and either: (i) at least partially optically transparent, or (ii) optically reflective with a different reflectance to the first material. The selected appearance property for the first subregion may comprise an overall reflectance value for the first subregion.

In an aspect, there is provided a touch sensor (e.g. a capacitive touch sensor) comprising a sensor array of sensor pixels, wherein: a first electrically conductive element in a first subregion of the sensor array is provided by a combination of both a first material and a second material; and the first material is electrically conductive and optically reflective, and the second material is electrically conductive and either: (i) at least partially optically transparent, or (ii) optically reflective with a different reflectance to the first material.

A first area of the first electrically conductive element may be provided by the first material and a second area of the first electrically conductive element may be provided by the second material. For example, the first and second area may form a reflective area for the conductive element, e.g. the reflective area may be formed of a first part (e.g. the first area) and a second part (e.g. the second area). The relative area contributions of each of the first and second material to the reflective area may be controlled to provide a selected reflectance value for that optically reflective element. The second material may be provided on a user-facing surface of the first material. The second material may not fully cover the user-facing surface of the first material. The second material may be more reflective than the first material. The second material may have an optical reflectance of at least 50%. The first electrically conductive element may be one of: (i) a sensing electrode (e.g. a capacitive sensing electrode) of one sensor pixel in the sensor array, and (ii) an electrical shield layer for one or more sensor pixels in the sensor array. The second material may be thinner than the first material. The second material may have a thickness of under 500 nm, for example under 300 nm, for example under 250 nm, for example under 200 nm, for example under 100 nm. For example, the second material may have a thickness of 300 nm or less. The sensor may comprise a second electrically conductive element in a second subregion of the sensor array.

The second electrically conductive element may be provided by a combination of the first and second materials. The second electrically conductive element may be provided by a different combination of the first and second materials to the first electrically conductive element. Each subregion of the sensor array may contain an electrically conductive element comprising a combination of the first and second materials. The electrically conductive element in at least one subregion may be either entirely provided by the first material or entirely covered by the second material. Each sensor pixel may be provided by a multi-layered pixel stack containing a plurality of electrically conductive layers. The first electrically conductive element may be provided in one of said electrically conductive layers. The sensor may comprise a colour filter layer above the first electrically conductive element.

In an aspect, there is provided a method of designing a touch sensor (e.g. a capacitive touch sensor) comprising a sensor array of sensor pixels, wherein the method comprises: obtaining an indication of a selected appearance for the sensor array; determining a required reflectance value for each subregion of the sensor array in order to provide the selected appearance for the sensor array; for each subregion of the sensor array, selecting a combination of a first material and a second material to be used to provide an electrically conductive element within the subregion so that an overall reflectance value for the subregion is the determined required reflectance value for that subregion; and outputting instructions containing the selected combination of first and second materials for said electrically conductive element in each said subregion of the sensor array.

The first material may be electrically conductive and optically reflective. The second material may be electrically conductive and either: (i) at least partially optically transparent, or (ii) optically reflective with a different reflectance to the first material. Obtaining the indication of the selected appearance for the sensor array may comprise obtaining a digital representation of the selected appearance for the sensor array. Determining a required reflectance value for each subregion of the sensor array may comprise: comparing the digital representation of the selected appearance for the sensor array to a spatial distribution of the subregions across the sensor array; for each subregion of the sensor array, identifying a corresponding area of the digital representation of the selected appearance for the sensor array which is to be represented by said subregion of the sensor array and determining a required reflectance value for said subregion to represent said corresponding area of the digital representation. Outputting instructions may comprise outputting computer instructions to be executed by a device to provide a sensor array comprising a plurality of said electrically conductive elements, where each element is to be provided by said selected combination of first and second materials.

In an aspect, there is provided a method of manufacturing a touch sensor (e.g. a capacitive touch sensor) comprising any method of designing a capacitive sensor (e.g. a capacitive touch sensor) disclosed herein, and wherein the method of manufacturing further comprising manufacturing a touch sensor (e.g. a capacitive touch sensor) comprising the selected combination of first and second materials for said electrically conductive element in each said subregion of the sensor array.

In an aspect, there is provided a precursor to a touch sensor (e.g. a precursor to a capacitive touch sensor) comprising a sensor array of sensor pixels, wherein: the precursor comprises a sensor stack comprising one or more electrically conductive layers; one of the electrically conductive layers of the sensor stack is provided by both a first material and a second material, wherein the second material is deposited on a user-facing surface of the first material.

The first material may be electrically conductive and optically reflective. The second material may be electrically conductive and either: (i) at least partially optically transparent, or (ii) optically reflective with a different reflectance to the first material. The precursor may provide an apparatus which, subject to a selective removal of first and/or second material therefrom could provide any touch sensor, e.g. any capacitive touch sensor, of the type disclosed herein.

In an aspect, there is provided a method of modifying such a precursor to provide a touch sensor (e.g. a capacitive touch sensor), wherein the method comprises removing some of the second material from the user-facing surface of the first material.

An amount of second material removed may be controlled to provide a selected reflectance value for each subregion of the sensor. The method may comprise removing some of the first material to provide a plurality of islands of first material, wherein at least some of the islands of first material have an area of second material on a user-facing surface thereof. Each island of first material may provide either: (i) a sensing electrode (e.g. a capacitive sensing electrode) of one sensor pixel in the sensor array, or (ii) an electrical shield layer for one or more sensor pixels in the sensor array. The area of second material retained on the user-facing surface of each island of first material may be chosen so that an overall reflectance for a subregion of the sensor array associated with said island of first material will be at a selected reflectance value.

Aspects of the present disclosure may provide one or more computer program products comprising computer program instructions configured to control operation of a sensor manufacturing assembly to manufacture any skin contact sensor (e.g. any capacitive biometric skin contact sensor) as disclosed herein and/or to control operation of a processor to design any skin contact sensor (e.g. any capacitive biometric skin contact sensor) disclosed herein.

### FIGURES

Some examples of the present disclosure will now be described, by way of example only, with reference to the figures, in which:

FIGS. 1a to 1c show schematic diagrams of capacitive biometric skin contact sensors.

FIG. 2 shows a schematic diagram of a capacitive biometric skin contact sensor.

FIG. 3 shows a portion of a sensor array of sensor pixels for a capacitive biometric skin contact sensor.

FIGS. 4a and 4b show example sensor pixel designs for a capacitive biometric skin contact sensor.

FIG. 5 shows a schematic diagram of a capacitive biometric skin contact sensor.

FIG. 6 shows a schematic diagram of a capacitive biometric skin contact sensor.

FIG. 7 shows a schematic diagram of a capacitive biometric skin contact sensor.

FIG. 8 shows a schematic diagram of a capacitive biometric skin contact sensor.

FIG. 9 is a schematic diagram of a capacitive touch sensor and Inset D of FIG. 9 shows a cross-sectional view of a pixel of the sensor.

FIG. 10 is a schematic diagram of a cross-sectional view of a capacitive touch sensor.

FIG. 11 is a schematic diagram of a cross-sectional view of a capacitive touch sensor.

FIG. 12 is a schematic diagram of a cross-sectional view of a capacitive touch sensor.

FIGS. 13a to 13g are schematic diagrams illustrating steps in a method of manufacturing a capacitive touch sensor.

In the drawings like reference numerals are used to indicate like elements.

### SPECIFIC DESCRIPTION

The present disclosure relates to the use of an optical colour filter in a capacitive biometric skin contact sensor. The capacitive biometric skin contact sensor includes a sensor array comprising a plurality of sensor pixels. Each sensor pixel has a plurality of different layers. This includes an optical colour filter layer, in which a colour filter has been deposited on another layer of the sensor pixel. The sensor is designed for light to pass through the colour filter layer towards a user of that sensor. The colour filter layer of each sensor pixel will filter certain light to provide a selected appearance for that sensor pixel, as seen by the user of the sensor. For this, the sensor may be provided in a transmissive colour filtering arrangement or in a reflective colour filtering arrangement (or a combination of both). For the transmissive colour filtering arrangement, the sensor may be ‘backlit’, so that a light emitter is arranged behind the sensor. The light emitter will direct light from behind the sensor, through the colour filter of the sensor, and to the user. For the reflective colour filtering arrangement, the sensor may include one or more reflective elements. The reflective elements are arranged beneath the colour filter. Light from a user side of the sensor (e.g. ambient) will therefore reflect off the reflective element and back to the user of the sensor, having also passed through the colour filter. A combination of the two arrangements may be provided in which the sensor is backlit and also includes at least one reflective element, so that both backlit light and reflected light may pass through the colour filter.

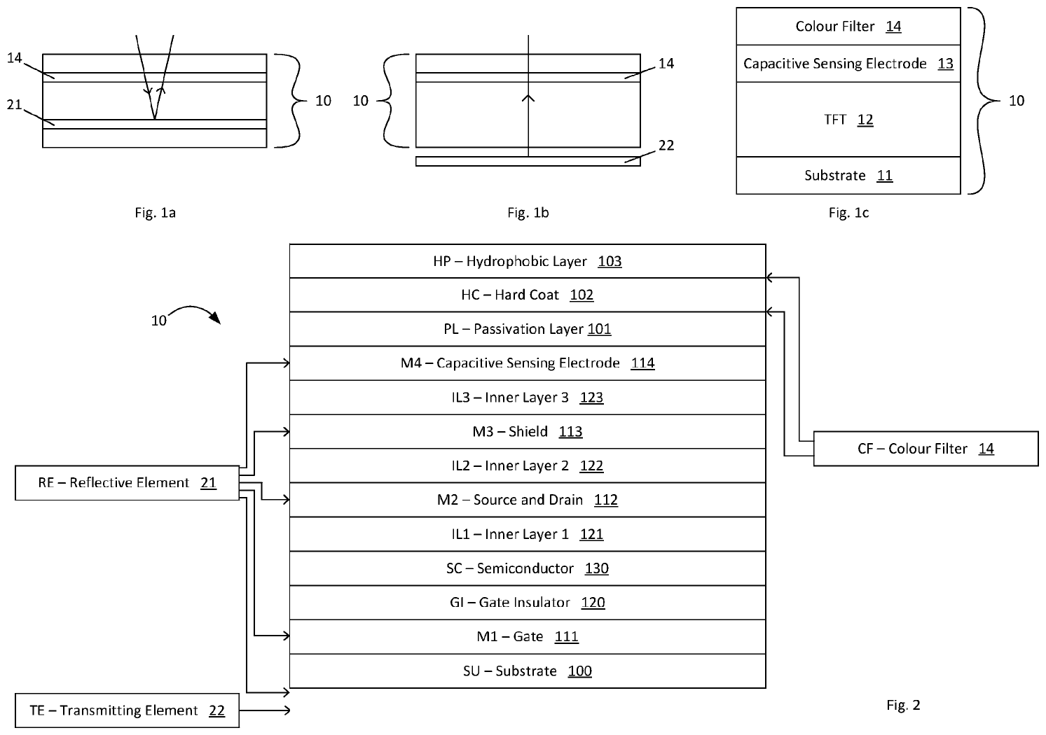

FIGS. 1a to 1c show example capacitive biometric skin contact sensors. Each of FIGS. 1a to 1c is shown in cross-section to illustrate different layers of the sensor.

The capacitive biometric skin contact sensors of the present disclosure include a sensor array formed of a plurality of sensor pixels. Each sensor pixel includes at least one thin film transistor (‘TFT’) and a capacitive sensing electrode. The sensor pixels are built up over multiple layers. For example, one layer may provide a substrate on top of which other layers of the pixels are stacked. In a ‘forward stack’, this substrate may be a base layer (on the opposite side of the sensor from the user) onto which the other layers are deposited. In a ‘reverse stack’, this substrate may be on a user-facing side of the sensor, and the other layers may be deposited onto a surface of this substrate facing away from the user of the sensor. The TFT may be provided over a plurality of different layers (e.g. to provide source/drain connections, a gate connection, and a semiconductor region). The capacitive sensing electrode is usually provided in one of the uppermost layers (so that it is closer to the user interacting with the sensor). For example, for a forward stack, the capacitive sensing electrode may be one of the last layers to be deposited, and in a reverse stack, the capacitive sensing electrode may be one of the first layers to be deposited. Additional examples of sensor pixel designs and sensor pixel stacks will be described in more detail below.

As will be appreciated, a user will interact with the sensor by placing a portion of their body into contact (or near to) the sensor. The sensor may provide a contact surface which the user will contact for sensing. For example, this may comprise the user placing their hand or finger (e.g. fingertip) on the contact surface. The capacitive biometric skin contact sensors of the present disclosure are configured to provide capacitive biometric skin contact sensing for that portion of the user's skin which is interacting with the contact surface. As such, a ‘user side’ of the sensor may be defined as the side of the sensor which is closest to the user of the sensor (e.g. the side of the sensor with which the user interacts, i.e. the side which provides the contact surface). An ‘opposite side’ may be defined as the side of the sensor which is furthest away from the user side of the sensor, i.e. the side of the sensor which is opposite to the user side of the sensor. For simplicity, the user side will be referred to as being ‘above’ the opposite side (and with the user themselves being ‘above’ the user side). However, it will be appreciated that this use of ‘above’ and ‘below’ does not require the sensor to always be provided horizontally with the user vertically above the sensor. Rather, the use of ‘above’ and ‘below’ is to describe whether features are closer to the user, or further away from the user (e.g. a first component will be closer to the user than a second component if the first component is described as being above the second component).

FIG. 1a shows a capacitive biometric skin contact sensor 10. The sensor 10 of FIG. 1a is a reflective colour filtering sensor. The sensor 10 includes a plurality of different layers. The sensor 10 includes an optically reflective layer 21 and an optical colour filter layer 14. The sensor 10 will also include additional layers (e.g. for including a capacitive sensing electrode and one or more TFTs), but these are not shown in FIG. 1a.

The optically reflective layer 21 is located beneath the colour filter layer 14. The optically reflective layer 21 comprises at least one optically reflective element. The optical colour filter layer 14 comprises one or more optical colour filters. The one or more colour filters may span a portion of each sensor pixel (e.g. they may cover the majority of a surface of each sensor pixel).

The optically reflective element is configured to reflect light incident on its top surface. In other words, the optically reflective layer 21 is configured so that, incident light that has travelled from a user-side of the sensor 10 (i.e. ambient light which travelled from above the optically reflective layer 21) will be reflected by the optically reflective element back towards the user (i.e. reflected upwards). The colour filter layer 14 is arranged above the optically reflective layer 21 so that light reflected by the optically reflective layer 21 will pass through the colour filter layer 14. The colour filter layer 14 will also filter light which travels from above the sensor 10 towards the optically reflective layer. This double filtering of light may reduce filtering requirements for the colour filter (e.g. a thinner layer of colour filter to be used), as light will pass through twice the thickness of colour filter. The one or more colour filters of the colour filter layer 14 are configured to filter light. Each individual colour filter may cause the light to appear a certain colour due to that colour filter filtering out other colours of light.

An example path for light through the sensor 10 is shown in FIG. 1a. The light travels from above the sensor 10 (on a user side of the sensor 10) towards the optically reflective layer 21. As can be seen, on this trajectory, this light will also pass through the colour filter layer 14 as it travels down towards the optically reflective layer 21. The light is then incident on a top surface of the optically reflective layer 21, where the optically reflective element causes reflection of that light. The reflected light then travels upwards and through the one or more colour filters of the colour filter layer 14, and then on towards the user of the sensor 10. This colour filtering will cause the sensor 10 to appear a certain colour to the user. That particular appearance will be controlled based on the choice of colour filter.

For FIG. 1a, the sensor 10 is arranged so that the optical colour filter of the optical colour filter layer 14 is located between: (i) the optically reflective element of the optically reflective layer 21, and (ii) a user interacting with the sensor 10. As such, the sensor 10 will provide optical colour filtering so that reflected light from the optically reflective element appears coloured to the user according to the one or more colours filtered by the optical colour filter layer 14.

FIG. 1b shows another example of a capacitive biometric skin contact sensor 10. The sensor 10 of FIG. 1b is a transmissive colour filtering sensor. The sensor 10 includes a plurality of different layers. The sensor 10 includes an optical colour filter layer 14. The sensor 10 will also include additional layers (e.g. for including a capacitive sensing electrode and one or more TFTs), but these are not shown in FIG. 1b. Also included is a transmitting element 22. The transmitting element 22 may be a backlight, e.g. a component which may illuminate the sensor stack from behind. The backlight may be part of the sensor 10. For example, the backlight may be provided by a component on a bottom surface (e.g. attached to a bottom layer) of the sensor 10. Alternatively, the backlight may be provided by another component located behind the sensor 10 (such as a light source), e.g. a component which is separate to the sensor 10. The optical colour filter layer 14 is provided as part of the sensor 10, i.e. it is provided as one of the layers within the multi-layer sensor stack.

The colour filter layer 14 of FIG. 1b may be similar to that described above in relation to FIG. 1a. The colour filter layer 14 of FIG. 1b may be thicker that of FIG. 1a, such as being double the thickness (e.g. as light may travel through the filter 14 in one direction, not two). The optical colour filter layer 14 includes one or more optical colour filters configured to provide optical filtering of light according to the selected colour(s) for that colour filter. The backlight is configured to direct light towards the colour filter. The backlight is arranged so that it will direct light through the colour filter and towards a user of the sensor 10. For example, the backlight may be a light emitting element (e.g. an LED, such as a white LED). In other words, the backlight may be provided by a component located underneath the colour filter (e.g. behind the colour filter, on a side of the colour filter opposite to the side on which the user interacting with the sensor 10 is located).

An example path for light travelling through the sensor 10 is shown in FIG. 1b. The light is emitted by the backlight (on the opposite side of the sensor 10 to the user) towards the optically reflective layer 21. As can be seen, on this trajectory, this light will pass through the colour filter layer 14 as it travels up towards the user of the sensor 10. This colour filtering will cause the sensor 10 to appear a certain colour to the user. That particular appearance will be controlled based on the choice of colour filter.

For FIGS. 1a and 1b, other components of the sensor pixel stack are not shown. In these Figs., the optical colour filter layer 14 is located so that light which reaches the user's eyes from the sensor pixel will have passed through the colour filter(s) of that pixel (and will thus appear coloured as per the one or more optical colour filters in that optical colour filter layer 14).

In relation to FIG. 1a, the sensor 10 includes an optically reflective layer 21 comprising an optically reflective element. The sensor 10 may include a plurality of different optically reflective layers, each comprising one or more optically reflective elements. The sensor pixel may be designed so that a majority of the surface area of the sensor pixel (when viewed from above) is covered by an optically reflective element. For example, the sensor pixel may be arranged so that a majority (if not all) of the surface area covered by the colour filter is also covered by one or more optically reflective elements (located beneath the colour filter).

Each optically reflective element may be provided by an existing component in the sensor pixel design. That is, the optically reflective element may be provided by using an optically reflective material to provide a component of the sensor pixel. In particular, electrically conductive materials may be used which are also optically reflective. That is, the electrical functionality of those components of the sensor pixel may not be altered, but those components may then also be optically reflective.

Each sensor pixel includes a capacitive sensing electrode. The capacitive sensing electrode for each sensor pixel will typically span a large surface area, e.g. the majority of the surface area (when viewed from above) of each sensor pixel. Each sensor pixel may also include one or more other conductive elements which span a large area of the sensor pixel, such as an electrical shield and/or a reference capacitor. Each sensor pixel may also include a plurality of electrical conductors for electrically connecting different components of the sensor pixel (e.g. for connecting capacitive sensing electrode to a gate region of the TFT etc.). Any or all of these different electrical components of the sensor pixels may be provided by an optically reflective material. Examples of optically reflective electrical conductors include Aluminium, Aluminium alloys (such as AlNd), titanium, gold, silver, molybdenum. For example, different combinations of materials could be used, e.g. one conductive layer of the stack may be formed from one material, and another layer in the stack may be formed from another material. Additionally, or alternatively, the sensor pixel may include one or more additional components, e.g. additional pieces of material, which have been included to provide the optical reflectivity for the sensor pixel.

An example capacitive biometric skin contact sensor 10 is shown in FIG. 1c. The sensor 10 is formed of a plurality of different layers. As shown in FIG. 1c, the sensor 10 includes a substrate 11, a TFT 12, a capacitive sensing electrode 13 and a colour filter 14.

The sensor 10 of FIG. 1c is a reflective colour filtering sensor. The colour filter 14 is provided on the top-most layer of the sensor 10 shown in FIG. 1c. However, it will be appreciated that additional layers may be included which are not shown, such as a hard coat and/or hydrophobic layer, which are provided on top of the colour filter layer 14.

The capacitive sensing electrode 13 is arranged to provide an optically reflective element for each sensor pixel. For example, the capacitive sensing electrode 13 may be made of an optically reflective aluminium alloy conductive material. The colour filter 14 for each sensor pixel may overlie the capacitive sensing electrode 13 for that pixel. That is, the capacitive sensing electrode 13 may be horizontally aligned with, and located under, the colour filter 14. The contact surface for the sensor 10 will be the top-most surface, so the colour filter 14 is located between the capacitive sensing electrode 13 and the user. The sensor 10 is arranged so that light which reflects off the optically reflective capacitive sensing electrode 13 may then pass through the colour filter 14 (to be optically colour filtered) before reaching the user's eyes.

For each sensor pixel, the capacitive sensing electrode 13 and the colour filter 14 may each span a majority of the cross-section (when viewed from above) of that sensor pixel. For example, this may maximise the aperture ratio for the colour filter (and e.g. to maximise the amount of filtered light output). As such, by using an optically reflective capacitive sensing electrode 13, and a colour filter 14 above that electrode 13, the desired colour filtering for the sensor 10 may be provided without the need to alter the sensor pixel design for the portion of the sensor 10 (and relevant components) located beneath the capacitive sensing electrode 13. Also, by placing the optically reflective element (in this case the capacitive sensing electrode 13) higher up in the sensor pixel stack, there are fewer intervening components which may attenuate the incident/reflected light.

As shown in FIG. 1c, the capacitive sensing electrode 13 may be the highest electrical component of the sensor pixel. The remaining electrical components of the sensor 10 are provided in layers beneath the capacitive sensing electrode 13. By providing the capacitive sensing electrode 13 in a higher layer, the capacitive response of the sensing electrode 13 to a conductive object (e.g. skin) contacting the contact surface of the sensor 10 will be greater (due to the decreased separation distance between the conductive object and the sensing electrode 13). Layers of the sensor pixel beneath the electrode 13 may also include conductive elements. For instance, in the TFT layer(s), which are below the electrode 13, there may be a plurality of different electrical conductors.

Some or all of the components in the lower layers of the sensor pixel may also be provided by an optically reflective material. For instance, any or all of the electrical conductors in the lower layers of the sensor pixel may be optically reflective. This may enable greater reflectivity for the light incident on the sensor pixel (from the user side of the sensor 10). As such, more light will be reflected back through the colour filter 14 and towards the user. For example, the components of the sensor pixel may be spatially arranged to maximise the coverage of optically reflective material across the sensor pixel. For instance, optically reflective elements (e.g. conductors) in layers beneath the sensing electrode 13 may be arranged to at least partially occupy regions of the sensor pixel which are not beneath the reflective electrode 13. In other words, the sensor pixel may be arranged to maximise the area of the sensor pixel that is covered with an optically reflective material.

As shown in FIG. 1c, the capacitive sensing electrode 13 is located above the TFT 12. The TFT 12 may span across several layers. A bottom most layer shown in FIG. 1c is the substrate 11 on which the other components of the sensor pixel are provided. One or both surfaces of the substrate 11 may have optically reflective material thereon. For example, at least a portion of (e.g. the entire) lowermost surface of the substrate 11 may be covered with an optically reflective material, and/or a separate reflective component could be provided beneath the substrate. This may further increase the amount of light which is reflected back through the colour filter 14 (especially in areas beneath regions of the sensor pixel which are not covered by the optically reflective electrode 13 and/or other optically reflective components of the sensor pixel). Also, by providing an optically reflective material on the opposite side of the substrate 11 (i.e. the side facing away from the user), this material may be less likely to interact with any components of the electronic circuitry of the sensor 10.

A larger sensor pixel stack will now be described with reference to FIG. 2.

FIG. 2 shows a sensor pixel stack for a capacitive biometric skin contact sensor 10. The stack shown in FIG. 2 includes more layers than those shown in FIGS. 1a to 1c. FIG. 2 is intended to show a number of options for different layers which could be included in the sensor pixel stack, but it will be appreciated that this stack should not be considered limiting. Some of these layers need not be included, and other additional layers not shown may also be included. Also, the particular ordering of the different layers should not be considered limiting. For example, the arrangement of the one or more TFTs of the sensor pixel could be altered (e.g. the source/drain and gate layers could be swapped). FIG. 2 is just intended to show a number of different example locations in a stack where a reflective element 21, transmitting element 22, and/or colour filter layer 14 may be included.

As with the example shown in FIG. 1c, the stack of FIG. 2 has a substrate 100. The substrate 100 forms a base of the stack on which other layers are provided.

The stack includes a plurality of layers which may carry one or more electrical conductors. These may be metallization layers. In the stack of FIG. 2, four metallization layers are included: (i) a first metallization layer (‘M1’), (ii) a second metallization layer (‘M2’), (iii) a third metallization layer (‘M3’) and, (iv) a fourth metallization layer (‘M4’). The M4 layer is the highest of the four, and the M1 layer is the lowest.

In the example stack of FIG. 2, the uppermost of these layers (M4) may be a capacitive sensing electrode layer 114, which may provide the capacitive sensing electrode for the sensor pixel. The next uppermost of these layers (M3) may be a shield layer 113, which may provide an electrical shield for the sensor pixel (e.g. to electrically shield the capacitive sensing electrode, such as from parasitic capacitances associated with components beneath the shield layer 113 in the stack). The two lowermost of these layers (M1 and M2) may be used to provide connections to one or more TFTs in the sensor pixel stack. In the example shown in FIG. 2, the M2 layer is a source and drain layer 112 and the M1 layer is a gate layer 111 (although the ordering for these layers could be reversed). The source and drain layer 112 may provide electrical connections to the source and drain regions of the one or more TFTs of the sensor pixel. The gate layer 111 may provide electrical connections to the gate region(s) of the one or more TFTs of the sensor pixel.

The electrically conductive layers (e.g. M1 to M4) may be separated from each other by additional layers of the sensor stack. These separating layers may be designed to electrically insulate components in adjacent metallization layers. A number of such insulating layers are shown in FIG. 2. These may include a gate insulator (‘GI’) layer 120, a first inner layer (‘IL1’) 121, a second inner layer (‘IL2’) 122 and a third inner layer (‘IL3’) 123. The stack may also include a semiconductor layer (‘SC’) 130 for the TFT(s). The stack may also be covered by a passivation layer (‘PL’) 101, a hard coat (‘HC’) 102 and/or a hydrophobic layer (‘HP’) 103.

The stack may be arranged with the substrate 100 as the base layer. The other layers are provided on top of the substrate 100. As shown in FIG. 2, the M1 gate layer 111 may be provided on the substrate 100. The M1 gate layer 111 may be separated from the semiconductor layer 130 by the gate insulator layer 120. The M2 source and drain layer 112 may be separated from the semiconductor layer 130 by the first inner layer 121. Although not shown, it will be appreciated that some of the layers may be connected to other non-adjacent layers. For example, one or more conductive vias may extend between the different layers, such as between the M2 layer and the semiconductor 130, or between the M4 layer and other M layers. The M2 source and drain layer 112 may be separated from the M3 shield layer 113 by the second inner layer 122. The M3 shield layer 113 may be separated from the M4 capacitive sensing electrode layer 114 by the third inner layer 123. The passivation layer 101 may be provided on top of the capacitive sensing electrode. The passivation layer 101 may comprise Silicon Nitride. A hard coat layer 102 may be provided on the passivation layer 101. A hydrophobic layer 103 may be provided on top of the stack.

The sensor pixel may be provided as a transmissive colour filtering pixel (which includes a transmitting element 22) or a reflective colour filtering pixel (with a reflective element 21). Where a reflective colour filtering pixel is used, the colour filter will be located above the reflective element 21 in the sensor 10. Where a transmissive colour filtering pixel is used, the colour filter will be located above the transmitting element 22.

The colour filter layer 14 may be provided in a plurality of different positions within the stack. The colour filter layer 14 may be provided by depositing optical colour filter material on another layer within the stack. The colour filter layer 14 may be provided on top of the passivation layer 101 (which covers the capacitive sensing electrode). The colour filter layer 14 may be provided on top of the hard coat 102.

The stack may be built up one layer at a time. Building the stack may involve sequentially building (e.g. depositing) layers on top of the substrate 100 and working up. For example, this would start by depositing the M1 gate layer 111 on the substrate 100, before depositing layers above that. Additionally, or alternatively, building the stack may involve building (e.g. depositing) layers onto the hard coat 102 or hydrophobic layer 103 and working down. For example, this may start by depositing the passivation layer 101 and then capacitive sensing electrode onto the hard coat 102. In some examples, when building down (e.g. from a hard coat 102), that layer may itself be temporarily affixed (e.g. glued) to a carrier substrate, which may then be subsequently removed later in the manufacturing process. For example, when building down, the layer (e.g. hard coat 102) onto which the subsequent layers are deposited may itself be a thin film. That thin film may be coupled to a carrier substrate to support it during the manufacturing process, before subsequently removing that carrier substrate.

In other words, the layers of the stack may each be built up sequentially (and separately). The colour filter layer 14 may be provided in the stack by depositing a colour filter onto the relevant layer during this process of building the stack. Depositing colour filter onto each sensor pixel may be done using photolithographic techniques. For example, the stack may be built up from the substrate 100 until the capacitive sensing electrode is provided, and the passivation layer 101 is deposited over the capacitive sensing electrode. The same process may be performed for all of the sensor pixels in the sensor array. Colour filters may then be deposited photolithographically over all of the different sensor pixels of the sensor array. The different sensor pixels may have different colour filters deposited thereon. A hard coat 102 and/or hydrophobic layer 103 may then be provided on top of the colour filter layer 14 (which would lie on top of the passivation layer 101 in this example). The colour filter layer 14 could be provided on top of other suitable layers, such as on top of the hard coat 102.

Where the stack provides a reflective colour filtering pixel, one or more of the existing layers in the stack may provide the optically reflective layer 21. The stack may include a plurality of optically reflective layers. Each optically reflective layer 21 may include at least one element which is optically reflective (so that light incident on that element from above is reflected back upwards towards the colour filter). Each optically reflective layer 21 may be provided by making the components in that layer out of an optically reflective material. For example, any of the metallization layers may utilise an optically reflective electrically conductive material, such as an aluminium alloy. For example, all of the metallization layers may be formed of an optically reflective material. The capacitive sensing electrode may be made of an optically reflective material. The electrical shield may be made of an optically reflective material. Any conductive elements in the M1 and M2 layers may be made of an optically reflective material.

Additionally, or alternatively, the optically reflective element 21 may be provided in a standalone optically reflective layer 21 to be included in the stack. Such an optically reflective layer 21 may be provided at any suitable location in the stack. For example, the optically reflective layer 21 may be provided on an under surface of the substrate 100 and/or as a separate layer beneath the substrate 100. That is, the surface of the substrate 100 on the opposite side of the user may be coated in an optically reflective material, and/or a separate reflective layer could be provided underneath the substrate 100.

Where the stack provides a transmissive colour filtering pixel, a transmitting element 22 may be provided. The transmitting element 22 may be in the form of a backlight for lighting the sensor stack from behind. The transmitting element 22 may be part of the sensor 10, or the transmitting element 22 may be a separate component to the sensor 10. For example, the transmitting element 22 may be a separate light emitting element onto which sensor stack is to be mounted. The transmitting element 22 may be a light emitter, such as an LED, which may be mounted onto an opposite side of the substrate 100 to the user. The transmitting element 22 may itself form the substrate 100 on top of which the sensor stack is provided. The transmitting element 22 may be arranged so that light may be transmitted, from the transmitting element 22, through the colour filter layer 14 (and towards a user of the sensor 10).

In these examples, each sensor pixel may be provided by a multi-layered stack. Within the layers of the stack, there will be at least one optical colour filter. Each sensor pixel is arranged so that some light will pass through that colour filter as that light travels towards a user of the sensor 10. The sensor pixel may therefore appear coloured to the user, where the colouring of the pixel is set by the particular colour filter chosen for that pixel (e.g. based on which colour(s) are used in the filter, and/or a ratio of colour filter to opaque, e.g. black, or transparent, e.g. white, colour filtering). In the reflective colour filtering pixel design, at least one of the layers in the sensor pixel stack will be an optically reflective layer 21 which includes one or more optically reflective elements. For example, any layers which contain a large coverage of electrical conductor, such as the M4 capacitive sensing electrode layer 114 or the M3 shield layer 113, may use an optically reflective electrical conductor to provide the one or more optically reflective elements for that layer. In the transmissive colour filtering pixel design, a transmitting element 22 is included, either as part of the sensor pixel stack, or as a component behind the stack. In either case, the result will be that light may pass through the colour filter of the sensor pixel towards the user.

In FIG. 2, there are a plurality of different layers of electrical conductors shown (M1 to M4). These layers may be provided by different materials. For example, for a reflective design, one or all of these layers may be made of an optically reflective material. The capacitive sensing electrode 114 (M4) may be made of a more optically reflective material than other layers. For example, the capacitive sensing electrode 114 (M4) may be made of an aluminium alloy, such as AlNd (or other suitable alloy) or silver, and/or M1 could be Molybdenum, M2 could be AlNd and/or M3 could be AlNd. One or more of the layers may be made of titanium, especially for lower reflective layers. For the transmissive design, M3 and/or M4 may be made of an at least partially transparent conductor, such as ITO. Non transparent components of the sensor, and/or relevant components within M1 and/or M2 layers may be aligned under a black colour filter component of each pixel.

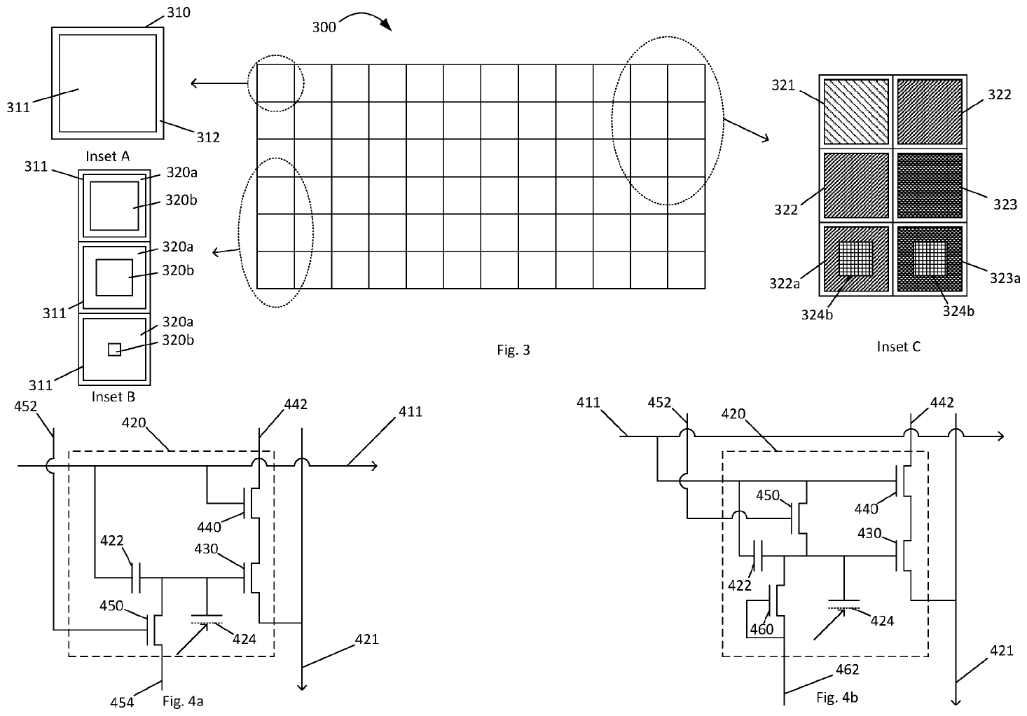

An example sensor array for a capacitive biometric skin contact sensor will now be described with reference to FIG. 3.

FIG. 3 shows a sensor array 300. The sensor array 300 is formed of a plurality of sensor pixels 310. The sensor array 300 may be a rectangular array comprising a plurality of rows of sensor pixels 310 and a plurality of columns of sensor pixels 310. Each sensor pixel 310 in the sensor array 300 may be similar. The sensor pixel design, as well as stack layup, for each sensor pixel 310 may be the same. However, the sensor pixels 310 may differ in the colour filtering they apply to their respective sensor pixel 310. The sensor array 300 may comprise an active matrix sensor array. For this, each sensor pixel 310 in a row may be connected to a gate drive channel for that row. Each sensor pixel 310 in a column may be connected to a read-out channel for that column. The sensor may be configured to apply a gate drive signal to one gate drive channel at a time. In response, each sensor pixel 310 in that row may output a read-out signal to the read-out channel for its row. The sensor may provide capacitive biometric skin contact sensing based on these read-out signals.

FIG. 3 shows a plan view of the sensor array 300. Inset A of FIG. 3 shows a zoomed in view of one sensor pixel 310 in the array 300. Each sensor pixel 310 is formed of two portions: a first portion 311 and a second portion 312. The first portion 311 is in a central region of the sensor pixel (e.g. the first portion 311 is a central portion of the sensor pixel 310). The second portion 312 is located around the edge of the sensor pixel 310 (e.g. the second portion 312 is a border portion). The second portion 312 may surround (e.g. completely circumscribe) the first portion 311. In other words, the first portion 311 occupies a central region of the pixel 310 and the second portion 312 occupies a perimeter region of the pixel 310.

Each of the first portion 311 and the second portion 312 may be covered by optical filtering material. For the first portion 311, this may comprise a colour filter (e.g. red, green, blue, transparent, e.g. white, opaque, e.g. black). For the second portion 312, the optical filtering material may filter substantially all colours of light. For example, the second portion 312 may utilise a black colour filtering material. The material which overlays the second portion 312 may be a light blocking material, such as a black colour filter. For example, the sensor array 300 may have a grid of black matrix material (e.g. a black colour filter which blocks light from passing therethrough), wherein the black material defines a series of rows and columns corresponding to the sensor pixels 310 of the array 300. In other words, this array of black matrix material may define a border region containing a black colour filter material for each sensor pixel 310 of the array 300. This border region may provide the second portion 312 of that pixel 310.

The first portion 311 and the second portion 312 may each provide respective colour filtering. The colour filtering provided by the first portion 311 will depend on what colour is used for the colour filter that overlays the first portion 311. The second portion will be overlayed by black colour filtering material, and so this will block light from passing therethrough. To maximise the transmission of colour filtered light through the first portion 311, the area of the first portion 311 within the sensor pixel 310 may be maximised. For example, the second portion 312 may provide a thin border around the edge of the pixel (relative to the area of the first portion 311 within a central region of that pixel). Surrounding each first portion 311 with the black material (e.g. with a black colour filtering second portion 312) may reduce the amount of light leakage associated with that pixel, which may enable a sharper static image to be shown by the sensor.

Each pixel may be square. Each pixel may be a 50×50 micron square. The first portion 311 of each sensor pixel 310 may be square, but is to be appreciated that this is just illustrative, the first portion 311 could be any shape. The first portion 311 may cover a majority of the sensor pixel area.

As described above, for each sensor pixel 310 of the sensor array 300, a colour filter layer 14 is included in the sensor pixel stack, and that colour filter will provide optical colour filtering of light passing through that colour filter. That colour filter will overlie the first portion 311 of the pixel 310. The sensor pixel 310 will then appear coloured, where the appearance of that sensor pixel 310 is dictated by the particular colour filter used. Such colour filtering may be provided for each sensor pixel 310 in the sensor array 300. In turn, this will provide a much larger area over which such colour filtering is performed, thereby giving rise to a much larger area of colour filtering. In this sense, each sensor pixel 310 of the sensor array 300 may also provide a pixel which influences the appearance of the sensor (e.g. due to the relevant colour filtering performed by that pixel 310).

The particular colour filter applied to each sensor pixel 310 may be selected to provide a selected overall appearance for the sensor array 300. For example, an image may be provided on the sensor array 300. The image will be a static image. The static image may be formed by the filtering performed by each colour filter of each sensor pixel 310. This includes colours, as per the different colours being filtered (e.g. R, G, B, W), as well as grayscaling for those colours (e.g. with K, black, filtering) As such, the sensor pixels 310 may effectively also provide pixels of the static image.

Each pixel of the static image will appear (e.g. will be coloured) according to the colour filter(s) of the corresponding sensor pixel 310 which provides that pixel of the static image. To provide a desired appearance for the sensor, an arrangement of colour filters may be chosen for each of the different sensor pixels 310 in the array 300 so that such an arrangement of colour filters will appear to provide a corresponding static image.

As will be appreciated in the context of the present disclosure, the particular appearance chosen for the sensor array 300 should not be considered limiting.

The colour filter(s) to be applied to the sensor pixels 310 may be chosen to provide colour matching. For example, the colour filter(s) to be applied to the sensor pixels 310 of the sensor array 300 may be selected to match surroundings to that sensor (e.g. to provide colour matching between the sensor array 300 and the surroundings to that sensor array 300, when installed in its intended location). The sensor array 300 may be monochromatic. Each sensor pixel 310 in the sensor array 300 may comprise the same colour filter. The sensor could be used to provide a touchpad, or a portion of a touchpad, (e.g. for a laptop). The sensor may provide capacitive biometric skin contact sensing functionality for that touchpad. Additionally, when the touchpad is installed, it may be surrounded by material (e.g. part of the laptop) which is all of a certain colour, and the colour filters applied to the sensor pixels 310 of the sensor array 300 may be colour matched to the colour of the material which surrounds the touchpad. That way, there may be a uniformity to the appearance of the device (e.g. laptop) which houses that touchpad (even though the touchpad may also provide capacitive biometric skin contact sensing functionality).

As described above, each pixel 310 may have a first portion 311 for providing colour filtering according to a selected colour, and a second portion 312 which blocks light around the perimeter of the pixel 310. The colour filters to be applied to the sensor array 300 may include more than one different colour. For example, the sensor array 300 could provide a static image, such as a photo, a logo, text (e.g. coloured text), by applying relevant different colour filters to the first portions 311 of individual sensor pixels 310 in the sensor array 300. Similarly, text may be shown on the sensor array 300 by using different colour filters for the sensor pixels 310 to show the writing. The colour filter material chosen may be selected to have certain thermal response properties. For example, a material may be used which changes colour when touched (e.g. in response to increased localised heating due to contact with a person's finger). As such, the sensor array 300 may be configured to respond to interaction with a user, such as to illuminate a certain portion of the sensor which was touched.

As will be appreciated, the sensor pixel resolution for the sensor array 300 will be much greater than that which the human eye can resolve. In other words, the human eye will not be able to resolve (unaided) the colour filtering being provided by each individual pixel 310 in the array 300. As a result, sensor arrays of the present disclosure may utilise only a few different colour filters for the sensor pixels 310 of the array 300. For example, blue, green, red, black and white (transparent) filters may be used, and these different colour filters may be combined to provide all of the different colours and shades for the sensor array. Blue, green and/or red colour filters may be combined to provide additional colour filtering (e.g. where the combination of two or more different colours makes another colour). Black and ‘white’ (transparent) colour filters may be combined with one or more other colours to provide grayscaling effects, e.g. to provide different shades of the same colour (by combining one colour with black/white to make a different shade of that colour). For example, variable amounts of black colour filter may be used to control how dark another colour appears (e.g. a higher proportion of black resulting in a darker colour).

Two examples of combining different colour filtering options will now be described with reference to Insets B and C in FIG. 3. Inset B shows an aperture ratio control technique being applied to the sensor pixels 310, and Inset C shows a pixel dithering technique being applied to the sensor pixels 310.

Inset B of FIG. 3 shows a column of three sensor pixels 310. For these three pixels, an aperture ratio for the colour filter in each pixel is varied. As described above, the sensor pixel 310 may have a first portion 311 (over which a colour filter may be placed) and a second portion 312 (over which a light blocking material may be placed). In Inset B, the first portion includes two different regions: first region 320a and second region 320b, and the second portion surrounds those two regions.

Within the first portion 311 of the pixel 310, two different colour filters may be applied (one to the first region 320a and one to the second region 320b). To provide maximum amount of colour filtering the full area of the first portion 311 of the pixel 310 is covered with a colour filter (R, G, B, W) and a minimum amount of black filter material is used (i.e. just that in the second portion 312 of the pixel 310). The first region 320a may be configured to filter a different colour to the second region 320b. In Inset B, the first region 320a is shown as surrounding the second region 320b. For example, the first region 320a may form an outer perimeter which surrounds an inner region (the second region 320b). The second region 320b may be a square which lies inside (the centre of) the first region 320a (which may have a square perimeter). However, other shapes may be utilised (e.g. circular, rectangular or other polygonal shapes), and/or the first and second regions could be arranged in a number of different ways with respect to each other.

The colour filter for the first portion 320a and the (different) colour filter for the second portion 320b may be selected so that they combine to provide a desired outcome colour. For example, by using two different colour filters, the sensor pixel 310 may appear according to a combination of those two colour filters. Any combination of colour filters may be provided. Alternatively, one of the first portion 320a and the second portion 320b will be covered with a black filter, and the other of the portions will be covered with a chosen colour filter. The ratio of the area of the first portion 320a relative to the area of the second portion 320b may be selected to provide a desired colour combination for the pixel 310. For example, where one of the portions is covered with black colour filter, by varying the ratio of pixel area covered by black filter to pixel area covered by other colour filter, a grayscaling (or darkness) may be chosen for that pixel.

This arrangement of varying the aperture ratio may find particular utility for providing colour grayscaling. For this, the aperture ratio for each pixel may be varied by utilising a black colour filter for one of the first and second regions of the sensor pixel 310. For example, the first region 320a may be black, and the second region 320b may be another filter colour (e.g. red, green, blue, transparent, e.g. white), or vice-versa. In examples where a black colour filter overlies the first region 320a, this black colour filter may be provided in combination with a black colour which overlies the second portion 312 of the sensor pixel 310. That is, as described above, the second portion 312 of each sensor pixel 310 may be overlayed by a black colour filter (such as black matrix material), and the border this second portion 312 provides around the colour filter may effectively be thickened so that this black colour filtering material also overlies the first region 320a of the first portion 311 of the sensor pixel 310. In other words, the first portion 311 of each sensor pixel 310 may represent the maximum amount of area per that pixel 310 to which a colour filter can be applied. As such, the maximum aperture ratio for each pixel 310 involves applying the colour filter(s) to cover the entirety of the first portion 311. This aperture ratio may be reduced by covering some of the first portion 311 with a black colour filter (i.e. effectively shrinking the area of the pixel 310 which is covered by a colour filter). The more this area is shrunk, the darker the light filtering provided by that pixel 310 will be.

For the sensor pixels in Inset B, the aperture ratio may be varied by varying the dimension of this black filtering portion of the pixel 310 (i.e. by varying the dimension of black colour filter material surrounding the colour filter of the pixel). In this sense, varying the dimension of the black filtering portion may comprise varying the proportion of the area which is covered by a black filter. For example, in Inset B, the first region 320a will be black and the second region 320b will be red (and also the second portion 312 which surrounds the first region 320a will also be black). The appearance (e.g. colour grayscale) of red will get darker down the column. In the topmost pixel shown in Inset B, there is not much black surrounding the red, and so the pixel would have a relatively strong red colour (a more saturated red colour). For the two pixels beneath it, there is an increasing proportion of black colour filter per pixel, and so these pixels will appear increasingly darker red (e.g. the red grayscale will be darker). For the lower pixels, there is a smaller aperture ratio, as more of the light will be filtered out by that black filter, and less will only pass through the red filter. In other words, the ratio of the area of the pixel 310 covered by black colour filtering material (e.g. light blocking material, as applied to the second portion 312 and the first region 320a) to the area of the pixel 310 covered by another colour filtering material (e.g. as applied to the second region 320b) is selected to provide a desired colour grayscale for that another colour.

In Inset B, different colour filters are applied within a single sensor pixel to provide a desired colouring effect for that pixel. Inset C shows an additional or alternative approach in which different colour filters are applied to different adjacent pixels to provide a desired colouring effect for a particular region of the sensor array.

Inset C shows a 2×3 grid of sensor pixels 310. A plurality of different colour filters are shown in inset B (four different colour filters are shown, but of course there could be more or fewer used). A pixel dithering technique is employed in which a desired overall appearance for a particular region of the sensor array is achieved by selecting colour filtering to be provided within each of a plurality of different pixels within that particular region. For instance, different colour filters may be used for different pixels within a cluster of pixels in the array, and the particular colour filtering to be provide by each pixel in that cluster is selected to provide a desired overall appearance for a region of the sensor array which is bigger than just one pixel.

To illustrate example pixel dithering functionality, four different colour filter colours are shown in Inset C (first to fourth colours, 321 to 324). Each pixel may also have black colour filtering provided in the second portion 312 which traces the perimeter of that pixel. The sensor pixels 310 are adjacent to each other, and they each individually cover a small area, so that a region of the sensor array 300 containing the 2×3 cluster of pixels will appear coloured according to a combination of the different colour filtering being performed by each pixel. Although a 2×3 grid is shown, it will be appreciated that this pixel dithering may be provided over a much larger area of the sensor array 300.

To provide pixel dithering, different colour filtering may be performed by different pixels within a cluster of pixels. For example, a first colour filter 321 may be applied to one pixel in the cluster, and a second (different) colour filter 322 may be applied to one or more adjacent pixels (two are shown as second colour filter 322 in Inset C). As the human eye may only resolve the colour filtering as provided by a larger area than the size of one pixel (i.e. a cluster of many pixels), colour filtering performed by each of a plurality of neighbouring pixels may effectively combine (e.g. to appear as though one single colour filter has been used). In other words, the cluster of pixels may appear according to a combination of the different types of colour filtering being performed by individual pixels within that cluster (e.g. according to first and second colour filtering).

By using such a pixel dithering approach, a greater number of different possible combinations of colour filters may be provided. For example, a higher resolution of colour grayscaling could be provided. That is, in order to provide a chosen colour (e.g. a chosen shade, or darkness etc.), different colour filters could be provided to each of a plurality of different neighbouring pixels so as to obtain the chosen colour when viewing the region as a whole. For example, within a particular region of the sensor, the amount of different colour filters applied can be varied, e.g. so that the ratio of different colour filters used within that region provides the desired overall appearance for that region as a whole. As an example, where varying the aperture ratio of a pixel (between black filtering and colour filtering) can vary the darkness for colour filtering by that pixel, the same effect may be achieved by instead applying the colour filter to some of the pixels (e.g. to the whole area of each said pixel) and applying the black filter to other pixels, where the ratio of colour to black filtering is the same for both (but this is imparted on a pixel by pixel basis rather than a sub-pixel basis. Of course, the two techniques could also be combined (as shown by the bottom two pixels in Inset C).

The pixel dithering may therefore provide more freedom for a selected static image of the sensor. For instance, within the small cluster of pixels, a third colour filter 323 could be used. Likewise, pixels may be provided which each contain two or more different colour filters (e.g. 322a and 324b, and 323a and 324b). For example, such pixels may utilise the variable pixel aperture ratio approach described above in relation to Inset B. Thus, the scope for varying the appearance of each region within the sensor array may be increased by providing a greater variety of colour filtering performed within a cluster of pixels in that region. That is, different colour filters may be applied to different pixels within a region, where the different colour filters applied (and the amount of pixels to which they are applied) are controlled so that the resulting combination from the different colour filtering performed by the different pixels, when viewing that region as a whole (as the human eye will), appears according to a selected appearance.

The pixel dithering or variable aperture ratio techniques may be used to provide blended transitions between different portions of the sensor array 300 (where different colours are filtered). For example, the pixels may extend from a first region where one colour filter is used through to a second region where a different colour filter is used. Between those two regions may be a transition region which includes pixels of both colour filters arranged to gradually change the colour from the colour of the first region through to the colour of the second region.

The pixel dithering or variable aperture ratio techniques may be used to provide colour grayscaling for the sensor array 300. For this, black or ‘white’ colour filters may be used for some of the sensor pixels 310 in an area (or for some regions within sensor pixels), and the other sensor pixels 310 in that area (or other regions of sensor pixels within that area) may be of one or more selected colour(s). A ‘white’ colour filter may comprise a transparent material, such as a fully transparent organic material (e.g. without colour). By varying the spatial density of black/‘white’ colour filtering in that area, a brightness for the overall colour seen in that region may be controlled. For example, for each cluster of pixels, there may be a certain proportion of black/‘white’ colour filtered pixels, as well as the relevant other colour filtered pixels (e.g. red pixels), and/or for each pixel there may be a certain proportion of black/‘white’ relative to said another colour. The darkness of that relevant other colour (e.g. red) may be controlled by selecting that spatial are of black colour filtering in that region (with more black pixels, and/or more black area, leading to a darker shade of that colour being shown). Likewise, by providing a greater proportion of ‘white’, i.e. a greater transparent area, the other colour being filtered may appear lighter.

For the pixel dithering approach, the same colour filter may be applied to more than one pixel in the same region. For example, different colour filters may be applied to clusters of pixels rather than just individual pixels. For example, a colour filter may be applied to each pixel in a 2×2 grid of pixels, and the arrangement of the different clusters of pixels (i.e. of different 2×2 grids of pixels) may be controlled to provide the desired colouring effect (e.g. to provide a desired colour or a desired grayscale effect for the colour in that area). Some pixels may not have colour filters at all. The number, and/or arrangement, of pixels without colour filters may be selected to provide a desired appearance property to the sensor.

It is to be appreciated in the context of the present disclosure that the different examples could be combined, or used interchangeably. For example, two different colours may be provided on the same pixel to provide another colour. Similarly, each pixel may have two different colour filters, but those pixels could also be used for pixel dithering with other adjacent pixel colour filters (which may have one or two colour filters). A pigment density and/or colour filter layer thickness may be selected to provide a desired saturation for the colour filtering (e.g. where increasing either will increase the colour saturation).

Additionally, or alternatively, and as will be described in more detail below with reference to FIGS. 8 to 13, one or more optical properties of the reflective element 21 may be modulated to provide a desired grayscaling effect on the colour filter. The reflectance of each individual pixel 10 may be controlled, and the reflectance value chosen for each pixel 10 may be such that it results in the desired colour filtering appearance for that pixel. For example, two differently reflective materials may be used to provide the reflective element 21. The contribution of each material to the overall reflectance for the pixel 10 may be selected so that the resulting reflectance value for that pixel 10 is a chosen reflectance value (e.g. thereby to provide a desired amount of grayscaling to a colour filter 14 covering that reflective element 21). As another example, the reflective element 21 may be formed of one at least partially optically transparent material and one reflective material. The amount of the reflective material covered by the transparent material may be selected to provide a desired property for the resulting reflectance from that reflective element 21.

In view of the above, a capacitive biometric skin contact sensor may be designed to have a selected appearance. For example, that selected appearance may be a particular colour (or a plurality of particular colours), or it may show a certain static image or message. Once the selected appearance for the sensor is known, a corresponding arrangement for the colour filters of the sensor array 300 may be determined. The colour filters may be chosen so that they will cause the image to appear according to the selected appearance due to the optical filtering of light passing through those colour filters to a user of the sensor.

The colour filters may be applied so that the colour filter layer 14 is less than one micron thick. The material for the colour filter may be selected to have a high dielectric constant. Additionally, or alternatively, the different colour filter materials for the colour filter layer 14 (e.g. for the different colours) may be matched. That way, the range/performance of each sensor pixel may be the same (despite different colour filtering). The capacitance to be sensed using the capacitive sensing electrode may therefore remain relatively unchanged as compared to a corresponding sensor pixel 310 which did not include a colour filter layer 14. Nevertheless, a calibration method may be performed for a sensor which has been manufactured to include a colour filter layer 14 in the sensor pixel stack. For example, an object with a known expected capacitive response may be sensed by the sensor. To the extent that the read-out from any of the sensor pixels 310 deviates from the expected values for those sensor pixels 310, a calibration may be applied to account for this deviation. For example, the sensor may comprise a controller which stores calibration data for read-out signals. The calibration data may be for some or all (e.g. for each) of the sensor pixels 310 in the sensor array 300. The calibration data may provide a mapping between an observed value, as measured using that sensor, and an adjusted value to which that observed value should correspond. For example, a black noise image calibration could be used. For this, the sensor may be operated to obtain capacitance data when no object is in proximity to the sensor array 300. The resulting capacitance data may be stored as a calibration data. That calibration data may then be used to calibrate subsequently obtained data (e.g. to subtract the influence of the black noise data from newly obtained data for an object). For example, this may facilitate calibration of the sensor to account for any differences in dielectric properties associated with the colour filter materials (e.g. differences between the R, G, B, W, K filter materials).

In examples described herein, a colour filter layer 14 is provided above a reflective element 21 and/or a transmissive element 22. That way, light travelling from said element towards a user of the sensor will be optically colour filtered according to the colour filter layer 14 in that pixel. As shown in Inset A of FIG. 3, each sensor pixel 310 may include a first portion 311 and a second portion 312. Light will be blocked by the second portion 312, so that light may only pass through the first portion 311 of the colour filter layer 14 (which may thus provide colour filtering thereof). The appearance of the sensor array 300 may be improved with a greater amount of light travelling through the colour filter (as well as a greater proportion of all light which reaches the user from the sensor travelling through a colour filter). The first portion 311 may take up a relatively large portion of the sensor pixel 310 (e.g. as large a portion as possible, except where aperture ratio techniques are applied with a greater proportion of black filter material provided to darken the apparent colour of that pixel).

Each sensor pixel 310 itself may be arranged to increase the amount of light which passes through the colour filter. For a transmissive colour filtering sensor pixel, light may take a path from the transmitting element 22 to the colour filter and through the colour filter to the user. For a reflective colour filtering sensor pixel, light may take a path into the sensor pixel 310 (from above) and through the sensor pixel 310 (and colour filter) until it reflects of a reflective element 21 of the sensor pixel 310, where that light will then be reflected and travel back upwards through the sensor pixel 310 (and colour filter) towards the user. For both types of sensor pixel 310, any non-transparent components of the sensor may be arranged so that they do not lie in these paths for light travelling through the sensor pixel 310. For example, any non-transparent components of the sensor may be provided in layers of the sensor pixel 310 beneath the reflective element 21 (or beneath the transmitting element 22). Additionally, or alternatively, any non-transparent components of the sensor may be provided underneath the second portion 312 of the sensor pixel 310 (where light will in any case be blocked, e.g. by black matrix material in the colour filter layer 14). As such, a greater proportion of available light may pass through the colour filter 14 (rather than being blocked by intervening components within the sensor pixel circuitry).

At least some of the components of the sensor could be provided by optically transparent materials. For example, electrical conductors could utilise a suitable transparent electrical conductor, such as Indium Tin Oxide (‘ITO’). This arrangement may enable light to pass through such conductive elements of the sensor without any substantial attenuation (e.g. with a minimal amount of the light being blocked). For example, the capacitive sensing electrode could be provided by a transparent material (rather than e.g. an optically reflective one). The colour filter layer 14 could for example be provided beneath the capacitive sensing electrode layer 114. The reflective or transmitting element 22 could be provided beneath the capacitive sensing electrode layer 114. For example, any non-transparent components, such as the one or more TFTs of the sensor pixel 310 could be located either beneath the transmitting/reflecting element(s) and/or underneath the second portion 312 of the sensor pixel 310. For example, in transmissive colour filter arrangements, the capacitive sensing electrode may be transparent (so too may be the electric shield, if included). Other non-transparent components (e.g. in M1 or M2) could be located beneath the second portion 312. For example, in the reflective colour filter arrangements, all of the layers (M1 to M4) may be provided by an optically reflective electrically conductive material.

In examples with a light transmitting element 22, the sensor may be configured to control operation of the light transmitting element 22. For example, the sensor may comprise a controller configured to selectively turn on or off one or more light emitting regions of the light transmitting element 22. The light transmitting element 22 may comprise a plurality of individual elements, each of which may be arranged to direct light towards a portion of the sensor array 300. The controller of the sensor may be configured to select which portion of the sensor array is illuminated by elements of the light transmitting element 22. For example, the controller may control operation of the transmitting element 22 so that some, but not all, of the sensor array is illuminated. This may mean that colour filtering only appears for a portion of the sensor array 300.

The sensor array 300 may be arranged so that the colour filters in different portions of the array 300 are different. The different colour filters may provide an indication of some information about operation of the sensor. For example, a green filter may indicate positive feedback (e.g. success at verification etc.), whereas a red filter may indicate an issue. The controller may be configured to selectively operate the light transmitting element 22 to direct light to a relevant portion of the sensor array. For example, in response to determining that biometric authentication was successful, light may be directed through a first portion of the sensor array (to use a first subset of the colour filtered pixels), and in response to unsuccessful biometric authentication, light may be directed through a second portion of the sensor array (to use a second subset of the colour filtered pixels). For example, the light may be directed through a certain subset of colour filters to indicate to a user that they should interact with that subset of sensor pixels in the sensor array.

The present disclosure may provide a capacitive biometric skin contact sensor formed of a plurality of sensor pixels, each having a colour filter layer 14 for providing a desired appearance to the sensor. The particular componentry for each sensor pixel should not be considered limiting. Typically, each sensor pixel will include a capacitive sensing electrode (for sensing proximity of a conductive object to be sensed) and at least one TFT (to give a read-out signal proportional to the capacitance sensed by the electrode). For example, the TFT may be operated to selectively provide a read-out signal indicative of the capacitance of the sensing electrode. The pixel may include a reference capacitor (or electrical shield) for shielding the sensing electrode from parasitic capacitances in the sensor array 300. The pixel may include other TFTs, such as a TFT for selectively activating individual sensor pixels, and/or a TFT for controlling biasing and/or reset circuitry for that pixel. The sensor array 300 may include a plurality of gate drive channels for providing gate drive signals to sensor pixels, and a plurality of read-out channels for receiving read-out signals from sensor pixels.

For such sensor pixels, the capacitive sensing electrode may be a first electrode (e.g. plate) which is arranged to effectively form a capacitor in response to proximity to the first electrode of a conductive body to be sensed (e.g. skin of a user, such as of their finger). By detecting changes in capacitance of the capacitive sensing electrode brought about by the proximity to the electrode of the conductive body, biometric data may be obtained for that conductive body. Biometric data may comprise any suitable biometric marker, such as an indication of the arrangement of ridges and valleys in the user's skin, and/or other skin markers such as sweat pores or glands etc.

Two example sensor pixel designs will now be described with reference to FIGS. 4a and 4b.

FIG. 4a shows a sensor pixel 420 for a capacitive biometric skin contact sensor. The sensor pixel 420 of FIG. 4a includes a capacitive sensing electrode 424 and a voltage-controlled impedance shown as a thin film transistor (‘TFT’) and referred to hereon in as ‘sense TFT 430’. The capacitive sensing electrode 424 is shown with a variable capacitor symbol. It will be appreciated that the capacitive sensing electrode 424 is formed of one electrode (e.g. a plate), and the variable capacitance for this will effectively be provided by a user interacting with that one electrode (e.g. due to the proximity of a portion of the user's skin proximal to the electrode). As will be appreciated, the capacitance associated with this capacitive sensing electrode 424 will vary in dependence on the proximity of the user's skin to the capacitive sensing electrode 424. The sensor pixel also includes a reference capacitor 422 (e.g. an electrical shield, as described above). For example, for the reference capacitor 422, the capacitive sensing electrode 424 may provide one plate of the reference capacitor 422, and an electrical shield layer 113 may provide a second plate of the reference capacitor 422. A first gate drive channel 411 is shown in FIG. 4a, as is a first read-out channel 421. The sensor pixel 420 of FIG. 4a also includes a select TFT 440, a select reference connection 442, a reset TFT 450, a first reset reference connection 452, and a second reset reference connection 454.

A first region of the sense TFT 430 is connected to the select TFT 440. A second region (e.g. a control terminal) of the sense TFT 430 is coupled to the capacitive sensing electrode 424. A third region of the sense TFT 430 is coupled to the first read-out channel 421. The second region of the sense TFT 4130 may be a gate region. The first region of the sense TFT 430 may be a drain region and the third region of the TFT 430 may be a source region. A first electrode of the reference capacitor 422 is coupled to the first gate drive channel 411. A second electrode of the reference capacitor 422 is coupled to the capacitive sensing electrode 424 and the second region of the sense TFT 430. As such, a connection between the second region of the sense TFT 430 and the capacitive sensing electrode 424 is also connected to the second electrode of the reference capacitor 422.

The select TFT 440 is coupled to the sense TFT 430 to selectively inhibit the sense TFT 430 from outputting a read-out signal to the first read-out channel 421. The select TFT 440 has a conductive channel connected in series between a reference signal supply and the sense TFT 430. A first region of the select TFT 440 is arranged to receive the reference signal supply (via the select reference connection 442). The second region of the select TFT 440 is coupled to the first gate drive channel 411, and a third region of the select TFT 440 is coupled to the first region of the sense TFT 430.

Additionally, reset circuitry is also coupled to the second electrode of the reference capacitor 422 (and thus the second region of the sense TFT 430 and the capacitive sensing electrode 424). The reset circuitry is configured to selectively tune the second region of the sense TFT 430 to a reference voltage (e.g. to provide a selected sensitivity for the pixel 420). A first region of the reset TFT 450 is coupled to the second electrode of the reference capacitor 422, the capacitive sensing electrode 424, and the second region of the sense TFT 430. A second region of the reset TFT 450 is arranged to receive a reset voltage (e.g. via the first reset reference connection 452). The first reset reference connection 452 may be connected to a preceding gate drive channel of the sensor. The reset circuitry is arranged so that, in response to the second region of the reset TFT 450 receiving the reset voltage, a conductive channel is opened between the first and third regions of the reset TFT 450. Current may flow either way through this channel (e.g. it could be arranged to permit current flow in either direction). For example, current may flow into the pixel 420 to charge the second region of the sense TFT 430 to a selected voltage (e.g. to tune its sensitivity), or current may flow away from the pixel to discharge the second region of the sense TFT 430. The second reset reference connection 454 thus connects the reset TFT 450 to provide relevant current flow (e.g. it is either connected to a reset reference voltage, or to distribute current elsewhere away from the pixel 420).

FIG. 4b shows a sensor pixel 420 for a capacitive biometric skin contact sensor. As with FIG. 4a, the sensor pixel 420 includes a capacitive sensing electrode 424, a reference capacitor 422 (e.g. an electrical shield layer 113 in combination with the capacitive sensing electrode 424), a sense TFT 430, a select TFT 440, a select reference connection 442, a reset TFT 450, and a first reset reference connection 452. As shown, the pixel 420 is connected to a first gate drive channel 411 and a first read-out channel 421. Also, the pixel 420 of FIG. 4b includes biasing circuitry comprising a bias TFT 460, and a bias reference connection 462.

The sensor pixel 420 shown in FIG. 4b is described in more detail in the Applicant's pending application GB 2013864.0. The structural arrangement of this sensor, the function of the sensor and the individual components of the sensor, and the method of operation as described in GB2013864.0 is incorporated herein by reference for all purposes.

The sensor pixels 420 of FIGS. 4a and 4b are similar in that they may each receive a gate drive signal from a gate drive channel which in turn gives rise to a capacitive potential divider arrangement involving the capacitive sensing electrode 424 and the reference capacitor 422. Likewise, this capacitive potential division controls operation of a TFT (sense TFT 430) to regulate the current output from the pixel in dependence on the proximity to the capacitive sensing electrode 424 of a conductive body to be sensed.

The sensor pixel 420 of FIG. 4b includes biasing circuitry comprising a one-way conduction path from a bias voltage connection to a control terminal of the sense TFT 430 so that current flows from the bias voltage towards the control terminal of the sense TFT 430 in response to the control terminal voltage of the sense TFT 430 dropping below a floor value. In other words, the biasing circuitry of the sensor pixel 420 is arranged to ensure that prior to making a measurement, the voltage at the control terminal (e.g. gate region) of the sense TFT 460 is at a selected value (e.g. a predefined voltage). The bias voltage may be varied to provide a selected voltage at the gate region of the sense TFT 430 (e.g. to provide a selected level of sensitivity for the sensor, or a define operation point for starting operation of the pixel). As shown in FIG. 4b, the biasing circuitry comprises a connection to the bias voltage (via bias reference connection 462) and the bias TFT 460. The bias TFT 460 is connected in diode configuration to provide the one-way conduction path. The drain of the bias TFT 460 is coupled to each of the second electrode of the reference capacitor 422, the capacitive sensing electrode 424 and the gate region of the sense TFT 430. As with FIG. 4a, the sensor pixel 420 of FIG. 4b includes reset circuitry selectively operable to provide a reference voltage on the reference capacitor 422. Similarly, the sensor pixel 420 of FIG. 4b may include select circuitry to selectively couple the sense TFT 430 to the supply voltage.

It will be appreciated in the context of the present disclosure that other sensor pixel designs could also be used. Also, sensor pixels have been described as being square, but it is to be appreciated that other shapes could be used. For example, the sensor pixel shapes may be selected with particular shapes or geometries based on the static image which will appear due to the colour filtering provided by those pixels. That is, the shape of each sensor pixel may provide the shape of each pixel of the static image, and so the sensor pixel may be shaped based on the desired display static image pixel shape. For example, the pixels may be triangular, rectangular, or any other suitable shape.

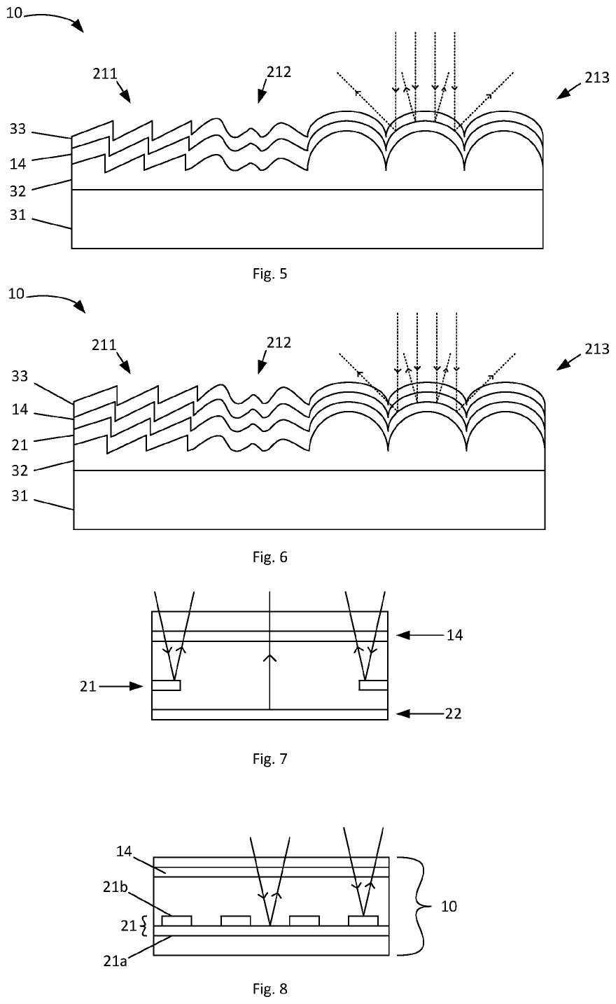

Further examples of capacitive biometric skin contact sensors will now be described with reference to each of FIGS. 5 to 7.

FIG. 5 shows a sensor 10. As with other examples described herein, the sensor 10 of FIG. 5 is formed of a plurality of different layers, one of which is an optically reflective layer 21. Also shown in FIG. 5 are lower layers 31 for the sensor, an intervening layer (shown as insulator layer 32) between the lower layers 31 and the reflective layer 21, and upper layers 33 for the sensor.

The optically reflective layer 21 is arranged to provide diffuse reflection of light incident on the layer 21 from above the sensor. Three different areas of the optically reflective layer are shown in FIG. 5: first area 211, second area 212, and third area 213. The three different areas are included just to show different reflective arrangements and functionality for the reflective layer 21.

As will be apparent, light incident on each of these three different areas will be diffusely reflected. For instance, some example light reflection trajectories are shown in FIG. 5 with dashed lines incident on the third area 213 of the layer 21. The layer 21 is arranged to provide inner diffuse reflection of ambient light. Ambient light incident on different portions of the layer 21 from directly above those portions will reflect in different directions (depending on which portion of the layer 21 the light is incident). For example, light incident on a certain portion of the layer 21 may reflect in a plurality of different directions. The optically reflective layer 21 may provide an at least partially curved mirror (e.g. where the curving is relative to a vertical axis). Diffusely reflected light may cause the sensor to appear less metallic to a user of the sensor (e.g. less shiny). For example, the sensor 10 may be arranged to provide diffuse reflection, thereby to have more of a matt appearance.

The optically reflective layer 21 has an uneven top surface. As shown, the layer 21 may pass over a series of bumps. The layer 21 may extend between a series of peaks and troughs, with the peaks being the vertically upper extents of the layer 21 (i.e. those which will be closer to a user of the sensor 10), and the troughs being the vertically lower extents of the layer 21 (i.e. those which will be furthest away from a user of the sensor 10). The layer 21 is not planar and horizontal. Instead, the angle which the top surface of the layer 21 makes relative to a vertical axis varies across the layer 21. For instance, light incident on the layer 21 from vertically above may reflect in a different direction when incident on different regions of the layer 21 (as those different regions of the layer 21 may be at different angles to the vertical). The changes in angle for the surface of the reflective layer 21 relative to the vertical axis may vary across the surface. These variations may repeat according to a selected pattern (e.g. a recurring/repeating pattern), or they may vary randomly across the surface. For example, the layer 21 may pass over a series of bumps to provide corresponding bumps (or ‘islands’) in the reflective layer 21. Those islands may provide the uneven top surface to the reflective layer 21. The pattern for the islands in the reflective layer 21 may be imparted by choosing a corresponding pattern for bumps in the layer onto which the reflective layer 21 is deposited.

The particular arrangement for the profile of the top surface of the reflective element 21 should not be considered limiting. The first, second and third areas shown in FIG. 5 each have very different profiles when viewed in cross section. That is, the profile of the top surface varies according to different patterns in each of these areas.

For the first area 211, the pattern is a repeating pattern with a bias direction for reflected light. For example, the first area 211 may be arranged to provide a chosen viewing direction for the sensor 10. That is, reflected light from the reflective layer 21 of the sensor 10 may be disproportionately directed in one direction. For example, this may find particular utility where a user of the sensor 10 will often interact with the sensor 10 from a fixed location/direction, and the reflected light may be disproportionately directed towards that direction. In FIG. 5, the first area 211 is shown with a saw-tooth profile, e.g. so that a majority of the incident light may reflect of the saw teeth portions (e.g. and thus in a desired direction).

For the second area 212, a random pattern is applied. This pattern may be neither regular nor repeating. Instead, the frequency at which different peaks and troughs may vary (e.g. it may be random). The height from each peak to its next trough may vary (e.g. it may be random). Incident light may reflect in a plurality of different directions, where the particular direction in which that light reflects depends on the particular point where that light is incident, and the angle which the top surface makes to the vertical at that point. Use of a random pattern may inhibit generation of unwanted optical artefacts, such as a ‘rainbow’.

For the third area 213, a regular repeating pattern is applied. This is shown as a series of consecutive bumps, where each bump has the same profile (e.g. the same shape and/or size), and the frequency with which each subsequent bump occurs is constant.

The examples of FIG. 5 are all shown in cross-section, where a side on profile for the layer 21 is described. However, it will be appreciated that the islands of material in the layer 21 will be three dimensional volumes. When viewed in plan those volumes may have selected shapes, and the profile for those shapes may vary in different regions. The shape of each island may vary across the reflective layer 21, and/or the frequency with which different islands occur in each direction may vary. Similarly, a size of each island may also vary from island to island (e.g. the area, when viewed in plan, of islands may differ). At least one of: a shape, profile, area, height, width, length, orientation of each island may be selected to provide a desired optical appearance. For example, any or all of these properties may be selected to provide a particular viewing direction for the sensor (e.g. to provide a biased reflection direction for the sensor). A spatial density of islands may be selected to provide a desired optical appearance. For example, each island may have a width and/or length of at least 2 microns, e.g. between 2 and 20 microns. For example, a spatial density of islands may be at least 2000 per square millimetre, such as between 2000 per square millimetre and 200000 per square millimetre.

As described above, sensors of the present disclosure may utilise one or more different optically reflective layers (e.g. to provide a reflector for the sensor). The arrangement shown in FIG. 5 could be provided for any suitable optically reflective layer. For example, an electrical shield layer, or a lower layer may be formed of a diffusely reflecting layer of the sensor (of the type shown in FIG. 5). One particular example for the different layers of the sensor will now be described with reference to FIG. 5.

The optically reflective layer 21 in FIG. 5 may be provided by the capacitive sensing electrode layer of the pixel. The sensor may include a plurality of lower layers (shown collectively as the lower layers 31 in FIG. 5) located beneath the capacitive sensing electrode optically reflective layer 21. These lower layers 31 may provide one or more TFTs, as well as other electrical connections, a shield layer and/or relevant insulator layers between adjacent conductive layers. Above the lower layers 31, and beneath reflective layer 21 is an intervening layer. The intervening layer may be an insulator layer 32 (such as IL3 123 of FIG. 2). The reflective layer 21 is on top of the insulator layer 32. One or more higher layers 33 (shown as a single layer are provided above the reflective layer 21). The higher layers 33 may include a passivation layer, a hard coat, and/or a hydrophobic layer.

The optically reflective layer 21 may be shaped (to provide diffuse reflection) by depositing that layer onto an uneven surface. For example, the layer 21 may be of a relatively consistent thickness across the pixel, but it may still extend between peaks and troughs. Those peaks and troughs may correspond to peaks and troughs provided in a layer onto which the reflective layer 21 is deposited. In other words, to impart the desired spatial properties into the reflective layer 21, those properties may be imparted into a layer onto which that reflective layer 21 will subsequently be deposited (and so the layer 21 will adopt those properties). In FIG. 5, the layer 21 (capacitive sensing electrode) may be deposited onto insulator layer 32. Prior to depositing the layer 21, a pattern may be provided in insulator layer 32. For example, a series of grooves may be etched into the insulator layer. The grooves may form troughs in the insulator layer 32, and regions of the insulator layer where no material has been removed may form peaks in the insulator layer 32. The troughs may be separated by islands of elevated material. The uppermost portions of those islands (e.g. the highest points) may provide the peaks. As such, the top surface of the insulator layer 32 may be uneven (e.g. where the angle which the top surface makes to a vertical axis varies).

The reflective layer 21 may be deposited onto the insulator layer 32 so that a top surface of the reflective layer 21 has a corresponding cross-sectional profile to the top surface of the insulator layer 32 (e.g. thereby to provide an uneven top surface for to layer 21). The higher layers 33 may then be deposited on top of the reflective layer 21. Alternatively, the pixel may be built up in a reverse stack in which grooves are etched onto an inner surface of one of the higher layers 33, and the reflective layer 21 is then deposited onto that inner surface.

Adjacent peaks may be separated by at least 2 microns, e.g. 5 microns, such as at least 10 microns (although the separation distance may be chosen depending on the lithography method used to manufacture the sensor). A difference in height between peak and trough is at least 1 micron, such as between 1 and 10 microns (although again the particular height may be chosen based on tooling used to provide ridges in the insulator layer. The unevenness of the layer 21 may follow a selected pattern or it may be random. The separation distance between adjacent islands (e.g. between adjacent peaks, or between adjacent troughs) may have smaller than a pitch for the sensor pixels (e.g. there may be a plurality of different islands on each sensor pixel).

As with other examples, components in layers above the reflective layer 21 may be made of transparent materials to increase the amount of reflected light passing therethrough. Similarly, once the sensor is manufactured, a calibration may be performed (e.g. a black noise calibration) to obtain calibration data for the sensor (e.g. to account for any differences in capacitance brought about due to the uneven surface). This may be of particular utility when the capacitive sensing electrode provides the uneven optically reflective surface.

The sensor 10 of FIG. 5 may appear less shiny and metallic, while still able to operate as a capacitive biometric skin contact sensor. Such a sensor may therefore have a different appearance without compromising on the operational performance of that sensor.

FIG. 6 shows a similar sensor to that of FIG. 5. The sensor 10 of FIG. 6 differs from that of FIG. 5 only in that the sensor 10 of FIG. 6 also includes a colour filter layer 14. For sensors of the present disclosure which include one or more reflective elements (i.e. reflective surfaces) in combination with one or more colour filters, such reflective elements may be arranged to provide diffuse reflection (e.g. as described above in relation to FIG. 5). Such an arrangement is shown in FIG. 6, where a colour filter 14 is provided in one of the layers above the reflective layer 21. As such, diffusely reflected light from the reflective layer 21 will be colour filtered by colour filter 14 (e.g. so that colour filtering of diffuse reflected light may be provided). This may provide a softer appearance to the sensor (e.g. one which appears less shiny and metallic), while still enabling colour filtering to be provided as described herein.

FIGS. 1a and 1b show optically reflective and optically transmissive sensors respectively. However, it will be appreciated that the present disclosure also provides sensors which utilise both aspects in combination. One such sensor is shown in FIG. 7.

FIG. 7 shows a sensor which has both reflective elements 21 and a light emitting element 22, as well as colour filter 14. The sensor may be arranged to maximise an amount of light which may pass through colour filter 14. For this, intervening components of the sensor located between the light emitting element 22 and the colour filter 14 may be made of transparent materials and/or they may be clustered to try to minimise the amount of light from the light emitting element 22 that they block. For example, such elements may be located around the perimeter of the pixel (e.g. where they may be located underneath a black filtering portion of the pixel). Optically reflective material may also be used in such regions to increase the amount of light reflected by the sensor pixel (thereby to provide additional light to that emitted from the light emitting element 22). In other words, each pixel may be arranged so that light passing through the colour filter 14 to the user will include both reflected light from the reflective elements 21 and transmitted light emitted from the light emitting element 22. The arrangement of the reflective elements 21 and the emitting element 22 may be selected to maximise the amount of filtered light leaving each pixel. In some examples, the reflective elements 21 may be arranged to provide diffuse reflection (e.g. so that light reflected from perimeter regions of the pixel may still pass through the colour filter).

FIG. 8 shows another example of an optically reflective sensor. The sensor of FIG. 8 is similar to that shown in FIG. 1a, except that the sensor shown in FIG. 8 utilises multiple materials to provide a desired optical property for the reflected light.

FIG. 8 shows a pixel 10 of a capacitive touch sensor. As with FIG. 1a, the pixel 10 of FIG. 8 includes an optically reflective element 21 and a colour filter 14. The optically reflective element 21 may comprise two separate materials: a first material 21a, and a second material 21b. Both the materials may be electrically conductive. The two materials may be electrically connected so that they form the same electrical component. For example, the electrical component may be a capacitive sensing electrode 114. The two materials may have different optical properties. The ratio of first material 21a to second material 21b may be selected to provide a chosen resulting optical property for the electrical component. In particular, at least one of the materials may be optically reflective. The amount of the other material included may be modulated to vary an overall reflectance provided by the reflective element 21. Both materials may be optically reflective (as shown in FIG. 8, where light reflects off both materials), or one material may be optically reflective and the other at least partially optically transparent. For each reflective element 21, incident light will reflect off that element, from either the first material 21a and/or the second material 21b. The more of the more reflective material there is forming the reflective element 21, the brighter the resulting reflected light from the optically reflective element 21. Likewise, the more of the less reflective material/optically transparent material there is forming the reflective element 21, the darker the resulting reflected light from the optically reflective element 21. A chosen brightness/darkness for the reflected light may be selected by a choosing a corresponding ratio of first to second material to provide a reflective area of the optically reflective element 21.

For capacitive sensors of the present disclosure, components of the sensor may be designed to provide a desired reflectance for the sensor. The sensor may utilise two materials having different optical properties. One of the materials is optically reflective, and the other material may be either at least partially transparent or reflective with a different reflectivity to the first material. By varying the coverage of the first and second materials, a resulting reflectance may also be varied. A desired reflectance for the entire sensor array may be implemented by choosing the amount of first and second material which will contribute to the reflectance of the sensor. For this, one of the two materials may be provided on top of the other material, and the area of the bottom material covered by the top material may be selected to provide a desired ratio of a visible portion of the first material to a visible portion of the second material. The two materials may be electrically connected so that they provide the same component of the sensor. The upper material may be sufficiently thin such that, the presence or absence of the upper material on the lower material may not materially alter the resulting electrical properties of that component. This arrangement may therefore enable an additional degree of freedom for designing the appearance of the sensor without compromising the ability of the sensor to function properly.

The following disclosure of using two electrically conductive materials to provide an optically reflective element of the sensor may apply to any sensor disclosed herein which utilises an optically reflective element 21. For example, any optically reflective element 21 disclosed herein may comprise two or more materials, as disclosed herein, to vary an overall reflectance provided by that reflective element 21.

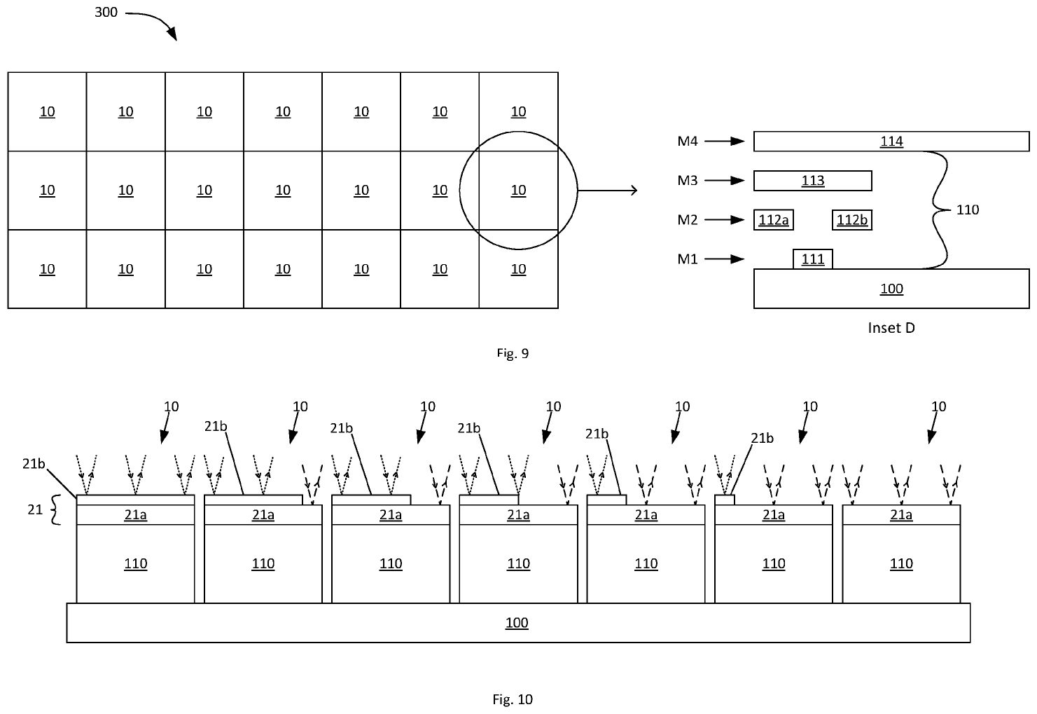

Example arrangements in which such first and second materials are incorporated into the sensor will be described later with reference to each of FIGS. 10, 11 and 12. An example arrangement for a sensor in which the two materials could be used will now be described with reference to FIG. 9. However, this is just one example, and the two materials could be used in combination with any of the sensors disclosed herein (such as the sensors of FIGS. 1a, 1c, 2 and 4 to 7).

FIG. 9 shows a sensor array 300 formed of a plurality of sensor pixels 10. The array 300 comprises a plurality of rows of sensor pixels 10 and a plurality of columns of sensor pixels 10.

Inset D of FIG. 9 shows a cross-sectional view of one of the sensor pixels 10 from the array 300. The pixel 10 shown in Inset D has four electrically conductive layers (e.g. metallisation layers) M1, M2, M3 and M4. To simplify the cross-sectional view, only the four metallisation layers and a substrate 100 are shown, but it will be appreciated that there may be may additional layers included (such as insulator layers between metallisation layers, one or more covering layers above the M4 layer, one or more semiconductor material regions etc.), and also the sensor pixel 10 could have fewer or more metallisation layers than those shown. Additionally, no electrical connections are shown between layers.

Each sensor pixel 10 may include at least one thin film transistor, TFT, and a capacitive sensing electrode. The sensor pixel 10 may also include an electrical shield. These example components are shown in Inset D of FIG. 9. For this, the first metallisation layer M1 includes a gate conductor 111, and the second metallisation layer M2 includes source and drain conductors 112a, 112b (such as the M2 layer 112 shown in FIG. 2). These conductors are respectively connected to gate, source and drain regions of at least one TFT. As such, a semiconductor region may be provided between the first and second metallisation layers M1 and M2. The third metallisation layer M3 includes a shield 113. The fourth metallisation layer M4 includes a capacitive sensing electrode 114. In the examples described below, the capacitive sensing electrode 114 may provide an optically reflective element 21, and this may be formed of two different materials. For simplicity when describing this arrangement, the layers beneath the capacitive sensing electrode 114114 (e.g. M1, M2, M3 and any intervening insulator and semiconductor layers) will be collectively referred to and shown as the lower layers 110 of the sensor pixel 10.

The capacitive sensing electrode 114 of each pixel 10 may be connected to a TFT of that pixel 10. For example, the capacitive sensing electrode 114 may be connected to the gate region of that TFT. The source and drain regions of that TFT may connect a read-out circuit and a reference signal (e.g. voltage) supply. One or more other TFTs may also be included in each pixel 10 for selectively controlling the application of the reference voltage supply to that pixel 10 and/or for resetting the pixel 10. For example, each pixel 10 may be of the type disclosed in either of the Applicant's earlier applications GB 2585420 (see e.g. the sensor pixel circuitry in FIGS. 1, 3, 5 and/or 6) and/or GB 2599075 (see e.g. the sensor pixel circuitry in FIG. 1).

The first and second metallisation layers M1, M2 may be used to provide the one or more TFTs of the sensor pixel 10. In FIG. 9, a bottom-gate configuration is shown, but a top-gate configuration (i.e. with M1 and M2 the other way round) could also be used. An optional third metallisation layer M3 is included in Inset D. The shield 113 of the third metallisation layer M3 is arranged to provide shielding of the capacitive sensing electrode 114 from electrically conductive components of the sensor. The shield 113 may be arranged so that it overlies all (or at least a majority of) the electrically conductive components of the sensor array 300 which are beneath the capacitive sensing electrode 114). In other words, the shield 113 may provide an intervening electrically conductive layer between the capacitive sensing electrodes 114 and electrically conductive components of the sensor pixels 10 in lower layers 110 thereof.

In operation, a user will interact with the sensor by contacting a contact surface of the sensor, and the sensor will provide capacitive touch sensing based on the contact from the user. A frame of reference for the sensor may be defined with respect to the user, so that the sensor has a ‘user-facing side’, and a side opposite to the user-facing side. The user-facing side of the sensor provides the contact surface for the user to contact. In the cross-sectional view in inset D, the user-facing side is a top side of the sensor (i.e. the sensor is arranged with a horizontal substrate 100 and the layers extending vertically upwards from the substrate 100 so that top layers are closer to the user than lower layers 110). The capacitive sensing electrodes 114 of the sensor array 300 are located in a layer close to the contact surface (e.g. a higher layer-typically they will be in the highest metallisation layer). For example, the contact surface may be a protective layer above the capacitive sensing electrodes 114.

Reference will now be made to FIGS. 10, 11 and 12, each of which shows a plurality of sensor pixels 10 in cross-section (i.e. when viewed side-on). In each of these Figs., the sensor pixels 10 are shown with a horizontal substrate 100 and with an uppermost layer of the pixel 10 being the closest layer to the user. In this regard, reference may be made to layers being ‘above’ or ‘below’ each other. However, it will be appreciated that this frame of reference relates to proximity to the user, rather than with reference to the vertical axis. For example, a higher conductive layer will be closer to the user than a lower conductive layer, but this does not mean that the higher layer must be vertically above the lower layer.

In each of FIGS. 10 to 12, at least some of the capacitive sensing electrodes 114 of the sensor array 300 are provided using two separate materials. A first of these materials is optically reflective. The second material has different optical properties to the first material. For FIG. 10, the second material is more optically reflective than the first material, and for FIG. 11, the second material is at least partially optically transparent. The overall reflectance provided is controlled by modulating the usage of the first and second material. For example, to increase reflectivity for each sub-region of the array 300, the more reflective material will provide a greater area of the user-facing side of the capacitive sensing electrode 114 within that sub-region. Likewise, to decrease reflectivity for each sub-region of the array 300, the more reflective material will provide a smaller area of the user-facing side of the capacitive sensing electrode 114 within that sub-region (i.e. the less reflective material will provide a greater area of the user-facing side of the capacitive sensing electrode 114 in that sub-region).

FIG. 10 shows a cross-section view of seven sensor pixels 10. As described above, each of the sensor pixels 10 comprises lower layers 110 on a substrate 100. For each sensor pixel 10, a capacitive sensing electrode 114 is provided on top of the lower layers 110 (i.e. on a user-facing side of the lower layers 110).

Each capacitive sensing electrode 114 comprises a first material 21a. Each capacitive sensing electrode 114 may also include a second material 21b. The different sensor pixels 10 shown in FIG. 10 have capacitive sensing electrodes 114 formed of different combinations of first and second material 21b. The left-most sensor pixel 10 in FIG. 10 has the greatest amount of second material 21b for its capacitive sensing electrode 114, and each pixel 10 to the right has less second material 21b, up to the right-most sensor pixel 10 which has no second material 21b. Therefore, at least some of the capacitive sensing electrodes 114 are formed of two materials: (i) the first material 21a, and (ii) the second material 21b.

The first material 21a and the second material 21b are provided in their own layers. In this sense, at least some of the capacitive sensing electrodes 114 are formed of two layers: a first material layer and a second material layer. The second material layer is the more user-facing of the two layers (i.e. the second material layer is provided above the first material 21a layer). For sensor pixels 10 without any second material 21b, the capacitive sensing electrode 114 may only comprise one layer (the first material layer).

Each capacitive sensing electrode 114 spans an area of the sensor array 300. For example, for each pixel 10 in the array 300, the capacitive sensing electrode 114 of that pixel 10 may span a majority of the area of that pixel 10. In other words, when viewed in plan, each capacitive sensing electrode 114 may cover its own respective area of the sensor array 300. Each capacitive sensing electrode 114 may cover the same amount of area. Each capacitive sensing electrode 114 covers its own respective portion of the sensor array 300 (e.g. so that the capacitive sensing electrodes 114 span across the area of the sensor array 300).

For each electrode 114, the area of that capacitive sensing electrode 114 may be covered by first and/or second material 21b. In other words, each capacitive sensing electrode 114 will have an area of material which will reflect light (hereinafter referred to as a ‘reflective area of the capacitive sensing electrode 114’). In other words, the capacitive sensing electrode 114 provides an optically reflective area formed of first material 21a and/or second material 21b. Each capacitive sensing electrode 114 may have the same reflective area. That is, each capacitive sensing electrode 114 may contain the same area of reflective material. The capacitive sensing electrodes 114 may differ in the material chosen to cover their reflective area. For example, the reflective area may be covered by the first material 21a, the second material 21b or any combination of the two materials.

For the left-most sensor pixel 10 in FIG. 10, the reflective area of the capacitive sensing electrode 114 is entirely covered by the second material 21b, and for the right-most sensor pixel 10 in FIG. 10, the reflective area of the capacitive sensing electrode 114 is entirely covered by the first material 21a. In other words, for the left-most pixel 10, the second material layer covers the entirety of the capacitive sensing electrode 114 for that pixel 10, and so the reflective area for that pixel 10 contains only the second material 21b. Conversely, for the right-most pixel 10, the first material layer covers the entirety of the capacitive sensing electrode 114 for that pixel 10 with no second material layer on top, and so the reflective area for that pixel 10 contains only the first material 21a. From left to right in FIG. 10, the reflective area of each pixel 10 contains increasingly less second material 21b.

For each pixel 10, the capacitive sensing electrode 114 comprises a first material layer covering the area of that electrode 114. For at least some of the pixels 10, the capacitive sensing electrode 114 also comprises a second material layer which covers at least a portion of the first material layer. The second material layer is located on top of the first material layer. Any portions of the first material layer which are covered by the second material 21b will not form part of the reflective area for that pixel 10 (as instead it will be the second material 21b on top which forms part of that reflective area). Likewise, portions of the first material layer which are not covered by a second material 21b will form part of the reflective area.

As described in more detail below, for each pixel 10, the area of the second material layer (i.e. the area of the capacitive sensing electrode 114 for which the first material 21a is covered by the second material 21b) is modulated to control an overall reflectance value provided by that capacitive sensing electrode 114. In other words, the proportion of first and second material 21a, 21b which contributes to the reflective area of the pixel 10 will be modulated.

Both the first material 21a and the second material 21b are electrically conductive. The two materials are electrically connected to each other. For example, the second material layer may be deposited on top of the first material layer, e.g. directly on top of the first material layer. The two material layers provide the same electrical component, which in the example of FIG. 10 is the capacitive sensing electrode 114.

The second material layer may be thinner than the first material layer. The majority of the electrical conductivity properties of the capacitive sensing electrode 114 may be imparted due to the first material layer. The second material layer may be relatively thin. For example, the second material layer may be thin enough that the presence or absence of second material 21b on top of the first material layer may not materially alter any resulting capacitance between the capacitive sensing electrode 114 and a conductive object to be sensed. In other words, the second material layer may be sufficiently thin that a capacitive sensing electrode 114 having a first material layer which is at least partially covered with a second material layer will obtain a similar (e.g. the same) capacitance measurement to a corresponding capacitive sensing electrode 114 having only a first material layer (i.e. with no second material covering). For example, the second material layer may have a thickness of under 5 microns, e.g. under 3 microns, e.g. under 2 microns, e.g. under 1 micron, e.g. under 500 nm, e.g. under 250 nm, e.g. under 100 nm.

Both the first material 21a and second material 21b are electrically conductive. With the second material layer deposited on top of the first material layer, the two will be electrically connected to form the same component. In other words, the first material layer and the second material layer are effectively shorted (i.e. electrically connected). The first and second materials 21a, 21b have different optical properties. In particular, the first material 21a will provide a different reflectance value to the second material 21b. In the example of FIG. 10, the second material 21b will be more reflective than the first material 21a. For example, the second material 21b may comprise a highly reflective material such as Aluminium or an alloy thereof. Other suitable materials could comprise e.g. Silver. The first material 21a will be less reflective. The first material 21a may have a relatively low reflectance value. In this sense, the first material 21a may be selected to provide a dark reflector and the second material 21b may be selected to provide a bright reflector. For example, the first material 21a may comprise a material such as Titanium, Tungsten Nickel and/or Platinum.

The sensor is arranged so that, when light is incident on the capacitive sensing electrodes 114 of the array 300, the brightness of the resulting reflected light is controlled by selecting the amount of first and/or second material 21b contributing to the reflective area of that pixel 10. The pixels 10 which provide the brightest reflection will be those where the second material layer covers the entirety of the capacitive sensing electrode 114 (i.e. where the reflective area of the pixel 10 only contains second material 21b). The pixels 10 which provide the darkest reflection will be those where there is no second material layer (i.e. where the reflective area of the pixel 10 only contains first material 21a). The ratio of first to second material 21b used to provide the reflective area of each pixel 10 may be varied to provide a desired overall reflectance value for that pixel 10.

The dashed lines in FIG. 10 show incident light on the capacitive sensing electrode 114 of each pixel 10. The smaller dashes represent brighter reflected light and the longer dashes represent darker reflected light. As shown, where light is incident on the second material 21b, the reflection will be brighter. In the left-most pixel 10, all of the light will be incident on second material 21b (as the second material 21b entirely covers the first material 21a), and so the reflection will be at its brightest. Moving rightwards, as the area covered by second material 21b decreases, an increasing amount of the incident light will be reflected from the first material 21a (rather than the second material 21b), and so the reflected light will be increasingly dark. By the right-most pixel 10, all of the incident light is reflected by the first material 21a and so this will be the darkest.

By varying the reflectance provided by different capacitive sensing electrodes 114 of the sensor array 300, an appearance property of the sensor array 300 may also be controlled. The overall appearance may comprise a constant reflectance value across the entirety of the array 300 (e.g. with each sub-region of the array 300 having the same reflectance value) or the reflectance value may vary in different sub-regions of the array 300.

The overall appearance property of the sensor array 300 may be provided by controlling an appearance property for each sub-region of the sensor array 300. Each sub-region may be provided by one capacitive sensing electrode 114, or it may be provided by a plurality of capacitive sensing electrodes 114 (e.g. neighbouring electrodes 114). As will be appreciated in the context of the present disclosure, the area and pitch for each capacitive sensing electrode 114 may be relatively small, and so to a human eye it may not be possible to clearly distinguish an individual reflectance of a single capacitive sensing electrode 114. Instead, the human eye may only be able to resolve an overall reflectance for each individual sub-region of the array 300, as provided by the reflectance of a plurality of capacitive sensing electrodes 114. To implement a certain overall appearance property for the sensor array 300, an appearance property may be controlled for each sub-region of the sensor array 300 (e.g. for each capacitive sensing electrode 114 and/or for each group of neighbouring capacitive sensing electrodes 114).

For each sub-region of the array 300, implementing the chosen appearance property for that sub-region comprises providing a selected reflectance value for that sub-region. The selected reflectance value for the sub-region comprises a reflectance in a range of reflectance values between a minimum reflectance and a maximum reflectance. The minimum reflectance is a reflectance value associated with using only the first material 21a in the reflective area of the one or more pixels 10 in the sub-region (i.e. with no second material 21b overlying the first material 21a). The maximum reflectance is a reflectance value associated with using only the second material 21b in the reflective area of the one or more pixels 10 in the sub-region (i.e. with the second material 21b overlying all of the first material). In other words, the selected reflectance value is between: (i) a darkest reflectance value, where only first material 21a is used to provide the reflector(s) in that sub-region, and (ii) a brightest reflectance value, where only the second material 21b is used to provide the reflector(s) in that sub-region. To provide any given reflectance within the range, the proportion of first to second material 21b is varied. For brighter reflectors, there will be more second material 21b present in the reflective area, and for darker reflectors, there will be more first material 21a present in the reflective area.

The reflectance properties may vary across the sensor array 300. For example, one sub-region of the array 300 may use more first material 21a in its reflectors than in another sub-region. One or more sub-regions may be at either the brightest or the darkest reflectance. By controlling the reflectance values for each sub-region of the array 300, an overall reflectance profile may be implemented for the entire sensor array 300. That overall reflectance profile may comprise a plurality of differently reflecting sub-regions or it may comprise uniformly reflecting sub-regions.

In other words, the overall appearance of the sensor array 300 (e.g. the reflectance profile for the array 300) may be implemented by varying the amount of second material 21b provided on each capacitive sensing electrode 114. The presence of second material 21b on capacitive sensing electrodes 114 may not materially alter capacitance measurements obtained by those electrodes 114, and so an overall appearance may be implemented for the sensor array 300 without requiring any modification to the operation of the sensor array 300.

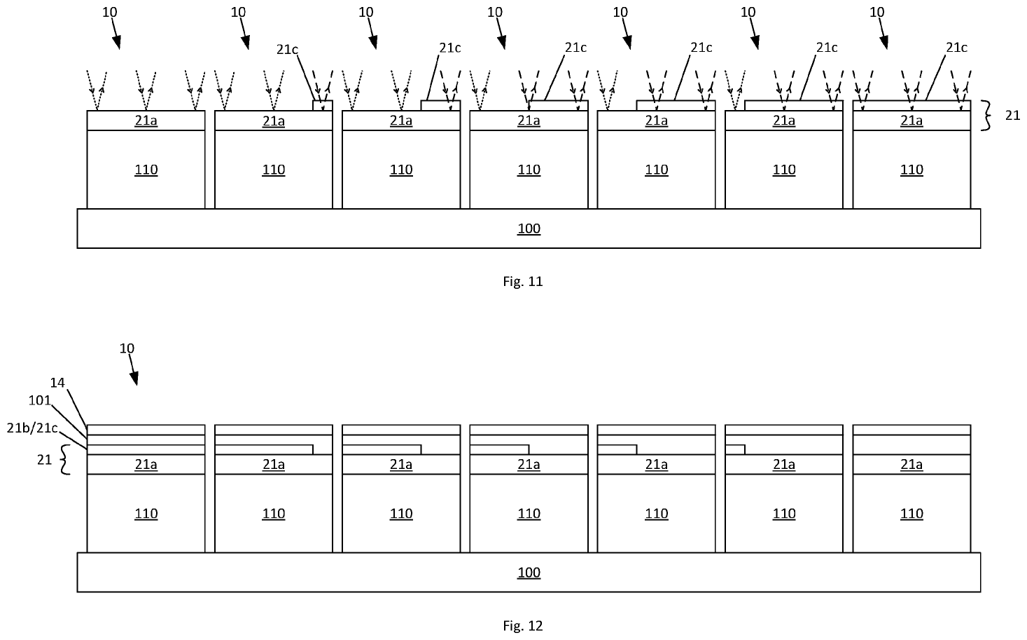

As described above, the present disclosure may comprise using an arrangement of colour filters to display a static image. For example, with different portions of the sensor array providing colour filtered areas which effectively form pixels of the static colour filtered image. The arrangement described above in which the overall appearance of the sensor array 300 is controlled by varying the amount of first or second material 21a, 21b in each sub region may also be used to provide a static image. In this sense, the differing reflectance values throughout the array 300 may contribute to providing a static monochromatic image. That is, without any colour filters included, the colour of the array may not vary, but the lightness/darkness may vary (e.g. due to the relevant ratio of first and second materials 21a, 21b). This variance in brightness may be used to generate a static monochromatic image. As will be described below in relation to FIG. 12, the two techniques may be employed together, with the reflection profile (e.g. brightness/darkness of reflected light) being varied by controlling the amount of first and second material 21a, 21b used in each sub-region, and the colour may be controlled by varying the choice and/or amount of colour filter used. This arrangement may therefore provide a static image which may contain multiple different colours (rather than a monochromatic one which may be provided without any colour filter layer at all).

Another example of a sensor will now be described with reference to FIG. 11.

FIG. 11 is very similar to the sensor of FIG. 10, and so like component parts will not be described again. As with FIG. 10, FIG. 11 shows seven pixels 10, where each pixel 10 comprises lower layers 110 with a capacitive sensing electrode 114 on top of the lower layers 110.

As with FIG. 10, in the sensor of FIG. 11, at least some of the capacitive sensing electrodes 114 are formed from two layers, with each layer containing a different material. A first material layer 21a in FIG. 11 may be much the same as the first material layer in FIG. 10. The first material layer may be a lower layer for the capacitive sensing electrode 114, and another layer may be deposited on top of (i.e. on a user-facing side of) the first material layer for at least some of the pixels 10. The sensor of FIG. 11 differs from that of FIG. 10 in that a third material 21c is used in the sensor of FIG. 11 instead of the second material 21b as used in FIG. 10.

The third material 21c is electrically conductive and at least partially optically transparent. The third material 21c may form an upper layer (i.e. on a user-facing side of the first material layer) for the capacitive sensing electrode 114, with the first material 21a forming a lower layer. As with the sensor of FIG. 10, the spatial coverage of the upper layer may be modulated to control an optical property for the capacitive sensing electrode 114. In the sensor of FIG. 11, the third material 21c is at least partially optically transparent (and thus not reflective), and so unlike with the sensor of FIG. 10, increasing the spatial coverage of material in the upper layer will not increase the reflectivity in that area. Instead, an area of the capacitive sensing electrode 114 which has first material 21a with third material 21c above it will provide lower reflectance than for an area where there is no third material 21c above. In this area, the reflected light may be less bright it may have a lower contrast ratio. The third material 21c may comprise any suitable electrically conductive and optically transparent material, such as Indium Tin Oxide (‘ITO’).

As will be appreciated, light incident on the capacitive sensing electrode 114 of the sensor will be reflected by the first material 21a. Where there is third material 21c above, the incident and/or reflected light will have to travel through the third material 21c. Where there is no third material 21c above, the incident and/or reflected light will not travel through third material 21c. This is shown by the dashed arrows in FIG. 11. The shorter dashes are for light which will be brighter and/or with greater contrast ratio than for the longer dashes. As can be seen the longer dashes are for light passing through third material 21c. From left to right in FIG. 11 the brightness and/or contrast ratio for reflected light is decreasing (as the capacitive sensing electrodes 114 contain more third material 21c).

As with the example of FIG. 10, a reflectance property for the capacitive sensing electrode 114, the sub-regions of the sensor array 300 and the array 300 as a whole can be controlled by modulating an amount of coverage of the third material 21c for each capacitive sensing electrode 114. As will be appreciated, the description above in relation to FIG. 10 also applies to the arrangement of FIG. 11, except that the amount of material in the upper layer (third material 21c) will be decreased in order to increase brightness (rather than increased as in FIG. 10). This description will not be repeated again here.

The overall appearance of the sensor array 300 (e.g. the reflectance profile for the array 300) may therefore be implemented by varying the amount of third material 21c provided on each capacitive sensing electrode 114. That is, by increasing the coverage of the third material 21c, the overall reflectance will be reduced, and by decreasing the coverage of the third material 21c, the overall reflectance will be increased. Again, the presence of third material 21c on capacitive sensing electrodes 114 may not materially alter capacitance measurements obtained by those electrodes 114, and so an overall appearance may be implemented for the sensor array 300 without requiring any modification to the operation of the sensor array 300.

Another example sensor will now be described with reference to FIG. 12.

The sensor of FIG. 12 includes the sensor of either FIG. 10 or FIG. 11. Additionally, for the sensor of FIG. 12, each pixel 10 may include a colour filter layer 14. For example, the sensor of either FIG. 10 or FIG. 11 may be included with a colour filter arrangement of the type disclosed in relation to any of FIGS. 1a, 1c, and/or 2 to 8. An optional layer (e.g. passivation layer 101) is shown in between the capacitive sensing electrode 114 and the colour filter layer 14 but this optional layer (e.g. passivation layer 101) could be omitted, and the colour filter layer 14 may instead be deposited directly on top of the capacitive sensing electrode 114. Where the optional layer (passivation layer 101) is included, it may be provided to provide a flat (e.g. planar) surface onto which the colour filter is to be deposited, or it may follow the same surface contours as for the layer(s) beneath it.

The sensor shown in FIG. 12 is a reflective colour filtering sensor. The capacitive sensing electrode 114 provides an optically reflective area for each pixel 10. The optical colour filter layer 14 provides optical colour filtering for each pixel 10. The reflective area (i.e. the capacitive sensing electrode 114) is located beneath the colour filter layer 14 (i.e. the capacitive sensing electrode 114 is located further away from the user than the colour filter). The optical colour filter layer 14 comprises one or more optical colour filters. The one or more colour filters may span a portion of each sensor pixel 10 (e.g. they may cover the majority of a surface of each sensor pixel 10).

The reflective area (the capacitive sensing electrode 114) is configured to reflect light incident on its top (user-facing) surface. In other words, the capacitive sensing electrode 114 is configured so that incident light that has travelled from a user-side of the sensor will be reflected by the optically reflective element back towards the user (e.g. reflected upwards in FIG. 12). The colour filter layer 14 is arranged above the reflective area (provided by the capacitive sensing electrode 114) so that light reflected by the reflective area of the capacitive sensing electrode 114 will pass through the colour filter layer 14. The colour filter layer 14 will also filter light which travels from above the sensor towards the reflective area (e.g. prior to being reflected). This double filtering of light may reduce filtering requirements for the colour filter (e.g. a thinner layer of colour filter to be used), as light will pass through twice the thickness of colour filter. The one or more colour filters of the colour filter layer 14 are configured to filter light. Each individual colour filter may cause the light to appear a certain colour due to that colour filter filtering out other colours of light. The particular appearance will be controlled based on the choice of colour filter.

The sensor is arranged so that the optical colour filter of the optical colour filter layer 14 is located between: (i) the reflective area of the capacitive sensing electrode 114, and (ii) a user interacting with the sensor. As such, the sensor will provide optical colour filtering so that reflected light from the reflective area appears coloured to the user according to the one or more colours filtered by the optical colour filter layer 14.

For each sensor pixel 10, a colour may be chosen for the colour filter(s) used in the colour filter layer 14 of that sensor pixel 10. An appearance of the sensor pixel 10 will therefore be influenced by the colour chosen for the colour filter. Additionally, the present disclosure may provide a further degree of freedom for selecting the colour of each sensor pixel 10 (or for each sub-region of the sensor array 300). That is, and as described above with reference to FIGS. 10 and 11, a reflectance value for each sensor pixel 10 may be modulated by varying the amount of second/third material 21b/21c used for the capacitive sensing electrode 114 of that pixel 10. For the example of FIG. 10, increasing the coverage of the second material 21b may increase a brightness of the reflection (whereas a greater coverage of the first material 21a may decrease the brightness), and for the example of FIG. 11, increasing the coverage of the third material 21c may decrease a brightness and/or contrast ratio for the reflection (whereas a smaller coverage of third material 21c may provide brighter and higher contrasting reflected light).

Sensors of the present disclosure may utilise these variable reflection properties to alter a resulting colour appearance for the colour filters used. That is, for a given colour chosen for the colour filter, the amount of second/third material 21b/21c used may be modulated to provide a selected brightness for that given colour. For example, where the colour filter is e.g. red, more second material 21b may be used to increase the apparent brightness of that red colour (or less second material 21b may be used to make that red appear a darker shade). As another example, in order to provide a fainter reflected image (with a lower contrast ratio), such as for a watermark, an increased amount of third material 21c may be used.

A selected overall appearance for the sensor array 300 may be provided by controlling: (i) the amount of second and/or third material 21b/21c used for each sensor pixel 10, and (ii) the choice of colour filter used for each sensor pixel 10. The choice of material used for the first, second and third material may also be selected to provide the selected overall appearance for the sensor array 300.

The selected overall appearance for the sensor array 300 may be uniform throughout the sensor array 300. For example, the entire sensor array 300 may be one particular colour. To provide that particular colour, a suitable colour filter may be selected and used for each pixel 10, and a corresponding reflection property for each pixel 10 may be selected and used. The reflection property may be selected so that the combination of the resulting light reflection and the colour filtering provided by the colour filter layer 14 provides the particular colour. For example, where the particular colour is a relatively bright shade, a greater area of second material 21b will be used for covering the reflective area of each capacitive sensing electrode 114, whereas where the particular colour is a relatively dark shade, a smaller area of second material 21b will be used for covering the reflective area. As another example, where the particular colour is fainter, an increasing amount of third material 21c will be used for covering the reflective area of each capacitive sensing electrode 114, whereas less third material 21c will be used for less faintly appearing colours.

The selected overall appearance for the sensor array 300 may not be uniform. The selected overall appearance may be formed of a plurality of sub-regions, where one or more of the sub-regions has a different appearance to other sub-regions. For example, the selected overall appearance could be in the form of a static image, e.g. a static photo, where the sensor pixels 10 or groups of neighbouring sensor pixels 10 effectively form image pixels of the static image. The static image may only be in a few colours (e.g. black and white, e.g. with grayscaling) or it may be a full colour image. Other examples for the selected overall appearance include providing a single colour area, where colour is uniform in that area, or incorporating indicia such as text and/or a logo into the appearance. As described above, each sub-region may contain one pixel 10, or it may contain a plurality of (neighbouring) pixels 10. For each sub-region, a particular appearance property (e.g. a particular colour) may be implemented by controlling the colour filter(s) used in the pixel(s) 100 in that sub-region, as well as controlling the amount of second/third material 21b/21c used in that sub-region. Each individual sub-region may therefore appear a particular colour, e.g. a particular choice of colour (as per the colour filter) and a particular brightness/contrast (as per the choices for material covering the reflective area). Each sub-region may have its appearance property controlled accordingly so that the overall appearance for the sensor array 300 may be the desired overall appearance (e.g. it may show the desired static image).

Controlling the appearance of a sub-region of the sensor array 300 may comprise controlling the colour filters and the material coverage for the reflective area for a plurality of pixels 10. Each pixel 10 within the sub-region need not have the same choice of colour filter and/or the same material coverage for its reflective area. These may be chosen for different pixels 10 so that the resulting appearance conforms to the desired appearance property for the sub-region. For example, the spatial resolution of the filters (e.g. the pixel resolution) may be higher than the human eye can resolve. In other words, the human eye may not be able to distinguish the colour filtering being provided by each different pixel 10. Combinations of pixels 10 may be used to provide a selected appearance for the sub-region. For example, the pixels 10 within the sub-region may have different colours and/or different material coverage of their reflective area, but where the resulting combination for those different pixels 10 provides the selected overall appearance for the sub-region.

The first material 21a may have relatively dark reflection properties (i.e. the resulting reflected light will appear very dark). Conversely, the second material 21b may have relatively bright reflection properties (i.e. the resulting reflected light will appear very bright). By increasing the area of the reflective area covered by the second material 21b, the brightness of the resulting reflected light will also increase, and the resulting colour filtering will give rise to a brighter and/or more vivid colour. Pixels 10 or sub-regions of the sensor array 300 in which the coverage of first material 21a dominates (e.g. where all, or a majority of, the reflective area is covered by the first material 21a) may be used to provide darker distinguishing areas of the array 300. For example, such pixels 10 could be used to provide a black matrix for the array 300 (e.g. regions of very dark reflected light which separate adjacent more brightly reflecting areas).

As will be appreciated in the context of the present disclosure, a sensor may be designed based on the desired appearance for that resulting sensor. The desired appearance may comprise an indication of how the sensor array 300 should appear (e.g. to show a static image/to maintain a constant colour etc.). Based on this desired appearance, a corresponding required appearance for each sub-region of the sensor array 300 may be determined. For example, this may comprise identifying what colour filter(s) to use in each sub-region (e.g. what colour filters to use for each pixel 10), and what reflectance property is required for each sub-region (e.g. what area of second/third material 21b/21c is to be used for covering the reflective area of each pixel 10).

Based on this, a requirements specification may be determined for the colour filters and reflective areas for each pixel 10 in the array 300. That is, it may be determined for each pixel 10 in the array 300: (i) what colour, if any, is to be used for the colour filter of that pixel 10, and (ii) what area of the reflective area for that pixel 10 should be covered by second or third material 21b/21c. The sensor may then be manufactured according to this requirements specification for the appearance of the sensor array 300, e.g. so that the resulting sensor conforms to the desired appearance for that sensor.

To implement a chosen appearance for the sensor, colour filters may be selected for each pixel 10. Additionally, the optical reflectivity for each optically reflective element may be controlled by modulating the materials used to provide each element (e.g. by selecting a ratio of first to second/third materials). Additionally, or alternatively, the aperture ratio for at least some of the pixels may be modulated, and/or one or more pixel dithering techniques may be employed to control an appearance of the sensor (e.g. so that a choice of colour and a brightness for the resulting optical colour filtering may be controlled).

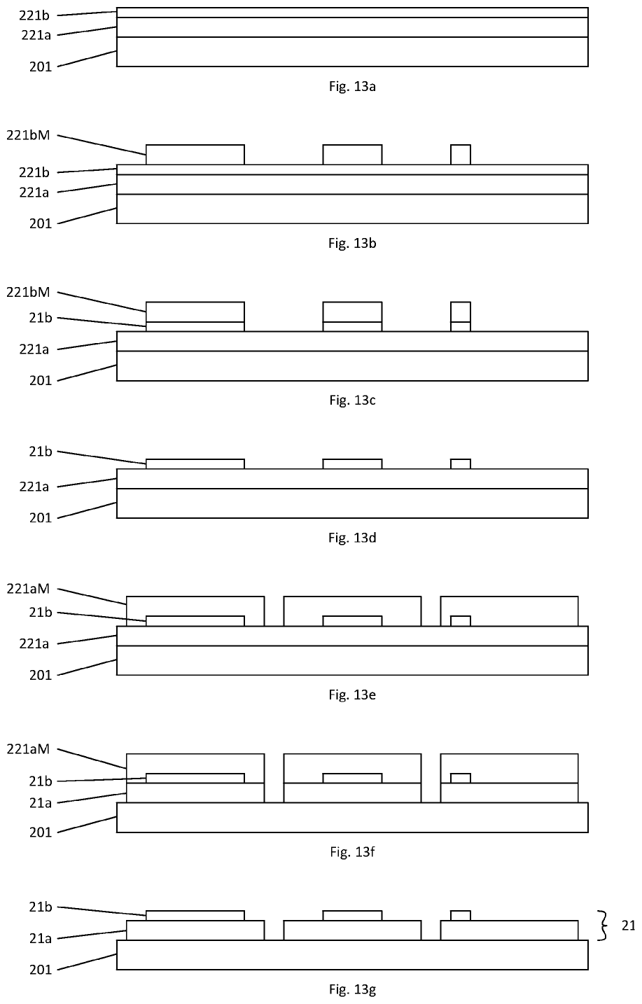

With reference to FIGS. 13a to 13g, an example method of manufacturing a sensor will now be described. As described in more detail below, this method may involve using photolithography to selectively remove electrically conductive regions of a precursor so that the remaining electrically conductive regions provide the desired first and second/third material coverage. For simplicity, this method will be described in relation to a sensor of the type shown in FIG. 10, i.e. which has a second material 21b (more reflective) rather than a third material 21c (transparent), but it will be appreciated that the method could just as well apply to sensors of the type shown in FIG. 11.

FIG. 13a shows a precursor. The precursor is arranged to be modified so that it may provide, after modification, a capacitive touch sensor. Only three layers are shown in the Figs. for the sake of simplicity, but it will be appreciated that many more may be present beneath (e.g. which provide lower conductive layers etc.). The lowest layer shown is a base layer 201. Above the base layer 201 is a first material layer 221a and above the first material layer 221a is a second material layer 221b. The first and second material layers 221a, 221b will be used to provide capacitive sensing electrodes 114 with first and second material 21a, 21b (as described above). The base layer 201 may comprise an insulating layer which separates the first material layer 221a from an electrically conductive layer beneath it in the pixel stack. The first and second material layers 221a, 221b are electrically conductive, and the base layer 201 is an electrical insulator. The base layer 201 may comprise Silicon Nitride. The first material layer 221a may comprise Titanium. The second material layer 221b may comprise Aluminium or an alloy thereof. The second material layer 221b is directly on top of the first material layer 221a (e.g. it may have been deposited onto the first material layer 221a).

The precursor shown in FIG. 13a may contain a plurality of layers, including a plurality of electrically conductive layers (e.g. metallisation layers), and wherein the uppermost electrically conductive layer is for the capacitive sensing electrodes 114. The portion of the precursor to be used to provide the capacitive sensing electrodes 114 is formed of two separate electrically conductive layers (first material layer 221a and second material 21b layer 221b).

In FIG. 13b, photoresist and masks are applied to be used for selectively removing some material from the precursor. Three masks 221bM are shown in FIG. 13b. The masks 221bM are arranged to control the removal of portions of the second material layer 221b. Each mask 221bM overlies the second material layer 221b. In particular, each mask 221bM is placed on a portion of the second material layer 221b which is to remain, while remaining portions of the second material layer 221b are to be removed. Each mask 221bM is therefore selected to provide the chosen amount of second material 21b to be used for each pixel 10. In FIG. 13b, three masks 221bM are used, with each mask 221bM being for a resulting pixel 10, and where the amount of second material 21b to be provided on the left-most pixel 10 is more than for the other pixels 10 (with the right-most pixel 10 having the least amount of second material 21b). The masks 221bM are applied to cover the desired portions of the second material layer 221b.

In FIG. 13c, the precursor is shown after a second material layer removal process has occurred. As shown, the portions of the second material layer 221b which were not covered by a mask 221bM have been removed, and the portions of the second material layer 221b which were covered by a mask 221bM remain. The removal process may comprise a selective etching process. The second material layer 221b may be substantially thinner than the first material layer 221a. Any etching away of the second material layer 221b may still leave the first material layer 221a substantially intact. The result of this second material layer removal process is that there are now islands of second material 21b on top of the first material 21a. Each island of second material 21b corresponds to a location where a mask 221bM was above the second material layer 221b. The resulting islands of second material 21b will form the portions of each capacitive sensing electrode 114 which are to be covered by second material 21b.

In FIG. 13d, the masks 221bM (and photoresist) have been removed from the second material layer 221b. As such, FIG. 13d shows three second material islands on the first material layer 221a. These three islands of second material 21b will provide the portions of second material 21b for the resulting capacitive sensing electrodes 114.

In FIG. 13e, photoresists and masks are again applied. Three masks 221aM are shown in FIG. 13e. The masks 221aM are arranged to control removal of portions of the first material layer 221a. In particular, the masks 221aM are used to separate out neighbouring capacitive sensing electrodes 114. Each mask 221aM may encapsulate the islands of second material 21b. For example, each mask 221aM may overlie and/or encapsulate any islands of second material 21b which are to be retained on each capacitive sensing electrode 114. The masks 221aM may all be of the same size and shape, e.g. so that the resulting capacitive sensing electrodes 114 are all of the same size and shape. Each mask 221aM may cover an area of first material 21a (and second material 21b for at least some pixels 10) which is to remain to provide the capacitive sensing electrode 114 of that pixel 10. The masks 221aM may be uniformly distributed across the array 300.

In FIG. 13f, a first material layer removal process has occurred. As shown, the portions of the first material layer 221a which were not covered by a mask 221aM have been removed, and the portions of the first material layer 221a which were covered by a mask 221aM remain. The removal process may comprise a selective etching process. The result of this first material layer removal process is that there are now islands of first material 21a on top of the base layer 201. Each island of first material 21a corresponds to a location where a mask 221aM was above the first (and second) material layer. Each resulting island of first material 21a will form the first material portion of the capacitive sensing electrode 114. At least some of the islands of first material 21a also have one or more islands of second material 21b above them (e.g. on a top surface thereof). The islands of second material 21b will form second material portions of the capacitive sensing electrodes 114.

In FIG. 13g, the masks 221aM (and photoresist) have been removed from the first (and second) material layer. As such, FIG. 13g shows three first material islands on the base layer 201 (and with each island of first material 21a having an island of second material 21b thereon). These three islands of first material 21a will provide the portions of first material 21a for the resulting capacitive sensing electrodes 114. The combined first and second material portions provide the capacitive sensing electrodes 114. Three capacitive sensing electrodes 114 are shown in FIG. 13g, each with first material 21a spanning the entire extent of the electrode 114, and each with at least a portion of second material 21b on top of an area of first material 21a. The resulting sensor therefore contains a plurality of capacitive sensing electrodes 114, and each electrode 114 may provide a selected reflectance property.

It will be appreciated in the context of the present disclosure that the examples described above are not intended to be limiting, but instead these provide certain examples for implementing the present disclosure. Other implementations are also envisaged. For instance, as described above, two electrically conductive material layers may be used to provide an optically reflective element of the sensor pixel. In the described examples, the element is a capacitive sensing electrode of the pixel. However, additional or alternative elements of the sensor could be used to provide the multi-layered optically reflective conductive element. For example, where a shield layer is used in the sensor pixels, that shield could be used as a reflective element. The shield may span across multiple pixels, and so it could provide a larger reflective area for the sensor array (as compared to that of an individual capacitive sensing electrode). Where the reflective element is provided by a component in a lower conductive layer of the sensor pixel (e.g. in a layer beneath the capacitive sensing electrode layer), some or all of the electrically conductive elements above may be at least partially optically transparent (e.g. made of ITO). Additionally, or alternatively, there may be more than one optically reflective element per sensor pixel. For example, these may be across multiple layers, e.g. both the shield layer and the capacitive sensing electrode layer could be reflective (as could other lower layers of the pixel stack). As will be appreciated, increasing the reflective area for a pixel (e.g. the area covered by reflectors, when viewed in plan) may increase an amount of light reflected.

Similarly, the layering shown in the Figs. need not be considered limiting. In the examples described, a layer of first material covers the entirety of the capacitive sensing electrode, with a variable amount of another material (second and/or third) on a top surface thereof. However, the ordering could be switched so that a variable amount of first material is provided on a top surface of a layer of second/third material. Likewise, there could be more than two conductive layers used to provide the electrically conductive component, e.g. second and third material layers could be provided on top of a first material layer, or another material altogether could be used. Alternatively, the first and second/third materials could be arranged interstitially within a single layer. For example, rather than one material being provided on top of another material, two separate materials could be used within the same layer (e.g. so that some portion(s) of the element were provided by a first material and other portion(s) were provided by a second material). When the second (more reflective) material is layered on top of the first material, and the second material is thin, this may advantageously reduce an amount of second material needed (which could be a more expensive material, thus reducing costs).

It will be appreciated that the pixel cross-sections should not be considered limiting, and these are only included to show possible examples to illustrate certain implementations of the present disclosure. For example, the pixel may have more or fewer conductive layers. The metallisation layers may contain non-metallic electrical conductors. A bottom gate configuration is shown in Inset D of FIG. 9 (i.e. with source and drain above gate), but a top gate configuration could also be used (i.e. with gate above source and drain). Likewise, the shield layer may not be included at all, or a single shield may span across a plurality of the pixels, e.g. so that the shield for a plurality of pixels is provided by the same component.

In examples where a colour filter is used, it will be appreciated that any suitable colour filter may be provided. The colour filter will be located above (i.e. on a user-facing side) of a reflective element. That way, the reflected light will pass through the filter for colour filtering thereof. The colour filters have been shown as separate layers, but the colour filter could be deposited directly on top of an existing component of the pixel stack, e.g. the colour filter layer could be deposited directly on top of the capacitive sensing electrode. Sensors need not include colour filters. As will be appreciated, appearance properties of the sensor may be implemented irrespectively of any colour filtering. For example, properties of the reflected light, such as brightness etc. may still be influenced by varying properties of the reflector (e.g. varying an amount of second/third material coverage there is for the reflective area of the pixel). An appearance of the sensor may therefore be controlled without the need for colour filters. It will also be appreciated that certain reflective materials will have wavelength-dependent reflection properties. That is, some materials may preferentially reflect certain wavelengths (i.e. colours). There may therefore be some colour preferencing occurring without the inclusion of a colour filter. For example, where ITO is used as a third material, this may provide a bias towards a more green colour. By modulating an area of second/third material on the reflective area, a colour bias of the reflectance may also be controlled.

As described above, an advantage of using two materials to provide the reflective element is that the provision of a thinner layer of electrically conductive material on top of the first material will not have any material impact on capacitance measurements obtained from the capacitive sensing electrode of the pixel. The sensor's appearance may therefore be controlled without influencing its ability to sense as intended. In any case, a black noise calibration could be performed for the sensors. For this, the sensor may be operated to sense nothing (i.e. with no conductive object proximal to the sensor array), and the obtained sensor data may be used in a subsequent calibration so that data obtained from each sensor pixel would give the same read-out value (i.e. that no conductive object is present). It will also be appreciated that, where reference is made to two separate materials being used, the two materials will have different optical properties, but they could be the same or a similar material (e.g. which has been modified/finished different so as to provide different resulting optical properties).

In examples described herein, sensors are provided for which one or more appearance properties can be varied by adjusting elements of the sensor. For example, the inclusion of one or more colour filter layers, a reflective element which provides internal diffuse reflection and/or in which elements of different reflectivity are used to provide a chosen reflectivity profile. In the examples described above, each sensor is a capacitive sensor, and the sensing electrode is a capacitive sensing electrode. The capacitive sensor may provide a capacitive contact (e.g. touch) sensor. The capacitive sensor may provide a capacitive biometric skin contact sensor. However, other sensor types may be used. For example, a sensor of the present disclosure may comprise an active matrix sensor

Additionally, or alternatively, the sensor may comprise a resistive sensor. The resistive sensor may be a resistive touch sensor. The resistive sensor may comprise one or more sensor electrodes which are used to sense contact. For example, the two sensor electrodes may be spaced apart, and contact with one electrode may cause that electrode to move and to bridge the gap between the two sensor electrodes, thereby electrically connecting them. A contact location may be determined based on this contact between the two sensor electrodes. For example, the resistive sensor may comprise two electrically conductive sheets separated from each other by a gap, such as an air gap. Each sheet may comprise one or more electrodes which are configured to apply a voltage profile across the sheet. For example, the voltage may decrease (e.g. linearly) from one electrode on one side of the sheet towards another electrode on the other side of the sheet. The second sheet may also comprise two electrodes which are configured to apply a voltage profile across the sheet from one electrode to the other. The electrodes may be arranged orthogonally. As such, this may provide a 2-D voltage profile (e.g. with one voltage profile extending along an x-axis, and another extending along a y-axis). The contact point may be determined based on the resulting voltage (e.g. the resulting voltage will provide an indication of where along the profile the contact is occurring). The sensor may operate first by obtaining a voltage readout from one axis (e.g. with the voltage profile being applied to a first of the sheets), before then obtaining a voltage readout from the other axis (e.g. with the voltage profile being applied to the other of the sheets). Thus, a first measurement may be obtained, where the measured voltage indicates how far along in one axis the contact point is. A second measurement may then be obtained, where the measured voltage indicates how far along in the other axis the point is. Based on these two axial coordinates, the position of contact may be determined.

It will be appreciated from the discussion above that the examples shown in the figures are merely exemplary, and include features which may be generalised, removed or replaced as described herein and as set out in the claims. With reference to the drawings in general, it will be appreciated that schematic functional block diagrams are used to indicate functionality of systems and apparatus described herein.

As will be appreciated by the skilled reader in the context of the present disclosure, each of the examples described herein may be implemented in a variety of different ways. Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus. In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Certain features of the methods described herein may be implemented in hardware, and one or more functions of the apparatus may be implemented in method steps. It will also be appreciated in the context of the present disclosure that the methods described herein need not be performed in the order in which they are described, nor necessarily in the order in which they are depicted in the drawings. Accordingly, aspects of the disclosure which are described with reference to products or apparatus are also intended to be implemented as methods and vice versa. The methods described herein may be implemented in computer programs, or in hardware or in any combination thereof. Computer programs include software, middleware, firmware, and any combination thereof. Such programs may be provided as signals or network messages and may be recorded on computer readable media such as tangible computer readable media which may store the computer programs in non-transitory form. Hardware includes computers, handheld devices, programmable processors, general purpose processors, application specific integrated circuits (ASICs), field programmable gate arrays (FPGAs), and arrays of logic gates.

Other examples and variations of the disclosure will be apparent to the skilled addressee in the context of the present disclosure.

1. A skin contact sensor comprising a sensor array of sensor pixels, wherein:
each sensor pixel comprises at least one thin film transistor, TFT, and a sensing electrode;
a top surface of the sensor array provides a contact surface for contacting by an object to be sensed; and
each sensor pixel is formed of a plurality of layers including an optical colour filter layer arranged to filter one or more colours of light.

2. The sensor of claim 1, wherein the plurality of layers includes an optically reflective layer located below the optical colour filter layer and comprising an optically reflective element.

3. The sensor of claim 2, wherein for each sensor pixel, the sensing electrode provides the optically reflective element.

4. The sensor of claim 2, wherein, for each sensor pixel, the optically reflective layer is a first optically reflective layer, and the sensor pixel also includes a second optically reflective layer comprising an optically reflective element, wherein the optical colour filter layer is located above the reflective elements of both reflective layers.

5. The sensor of claim 1, wherein each sensor pixel comprises an electrical shield layer comprising an electrical shield.

6. The sensor of claim 5, wherein the sensing electrode is located above the electric shield.

7. The sensor of claim 6, wherein both the sensing electrode and the electric shield provide optically reflective elements for each sensor pixel, and wherein the optical colour filter layer is located above the sensing electrode and the electric shield.

8. The sensor of claim 1, wherein all electrically conductive components of the sensor pixel provide optically reflective elements.

9. The sensor of claim 1, wherein, for each sensor pixel, the optical colour filter layer is provided on a top surface of the sensing electrode.

10. The sensor of claim 1, wherein, for each sensor pixel, a passivation layer is provided on a top surface of the sensing electrode, and wherein the optical colour filter layer is provided on a top surface of the passivation layer.

11. The sensor of claim 1, wherein, for each sensor pixel, a hard coat is provided on top of the sensor pixel, and wherein the optical colour filter layer is provided on a top surface of the hard coat or the hard coat is provided on a top surface of the optical colour filter layer.

12. The sensor of claim 1, wherein for at least some of the sensor pixels, an aperture ratio for the colour filter is varied to provide a selected property for the colour filtering provided by said pixels.

13. The sensor of claim 1, wherein, for at least some of the sensor pixels, the colour filter layer for the sensor pixel includes a first colour filter and a second colour filter, and wherein the first colour filter is for a different colour to the second colour filter.

14. The sensor of claim 1, wherein at least one of:
the sensor includes an optically reflective layer located below the optical colour filter layer and comprising an optically reflective element, wherein any non-transparent components of the sensor pixel in layers between the optically reflective layer and the colour filter layer are spatially arranged to inhibit blocking of light travelling between the optical reflective layer and the optical colour filter layer; and
the sensor is arranged to be backlit by a transmitting element located below the optical colour filter layer, and wherein any non-transparent components of the sensor pixel in layers between the transmitting element and the colour filter layer are spatially arranged to inhibit blocking of light travelling between the transmitting element and the optical colour filter layer.

15. The sensor of claim 14, wherein said non-transparent components of the sensor pixel are arranged to be laterally offset from an area of the sensor underneath the colour filter.

16. (canceled)

17. The sensor of claim 2, wherein each sensor pixel comprises at least one of:
(i) an optically reflective electric shield;
(ii) optically reflective source and/or drain conductive elements;
(iii) an optically reflective gate conductive element;
(iv) a substrate onto which the sensor pixel is built, wherein a surface of the substrate is optically reflective;
thereby to provide the optically reflective element of the sensor pixel.

18. An apparatus comprising the sensor of claim 1 and a light transmitting element, wherein the light transmitting element is arranged beneath the colour filter layer of the sensor pixels of the sensor.

19. A method of manufacturing a sensor, the sensor comprising a sensor array of sensor pixels, wherein a top surface of the sensor array provides a contact surface for contacting by an object to be sensed, and wherein each sensor pixel comprises at least one thin film transistor, TFT, and a sensing electrode, the method comprising:
for each sensor pixel, providing a plurality of layers for that sensor pixel including an optical colour filter layer.

20. The method of claim 19, wherein providing the optical colour filter layer for the sensor pixels of the array comprises use of a photolithography method.

21. (canceled)

22. (canceled)

23. A method of designing a sensor, the sensor comprising a sensor array of sensor pixels, wherein a top surface of the sensor array provides a contact surface for contacting by an object to be sensed, wherein each sensor pixel comprises at least one thin film transistor, TFT, and a sensing electrode, and wherein each sensor pixel is formed of a plurality of layers including an optical colour filter layer arranged to filter one or more colours of light, the method comprising:
obtaining an indication of a selected appearance for the top surface of the sensor; and
selecting at least one optical property for the optical filter of each sensor pixel of the array based on the selected appearance for the top surface of the sensor.

24-137. (canceled)
